(19) United States
(12) Patent Application Publication
(10) Pub. No.: US 2006/0127901 A1
(43) Pub. Date: Jun. 15, 2006

(54) Sim2 polypeptides and polynucleotides and uses of each in diagnosis and treatment of ovarian, breast and lung cancers

(75) Inventors: Chen Chermesh, Mishmar HaShiva (IL); Shira Walach, Hod-HaSharon (IL); Galit Rotman, Herzlia (IL); Osnat Sella-Tavor, Kfar-Kish (IL)

Correspondence Address: Martin D Moynihan;Prtsi, Post Office Box 16446, Arlington, VA, 22215, US

(73) Assignee: Compugen Ltd., Tel Aviv (IL)

(21) Appl. No.: 10/522,758

(22) PCT Filed: Aug. 3, 2003
(86) PCT No.: PCT/IL03/00636

§ 371 Date: Oct. 18, 2005

Related U.S. Application Data
(60) Provisional application No. 60/400,131, filed on Aug. 2, 2002.
(60) Provisional application No. 60/452,681, filed on Mar. 7, 2003.

(51) Int. Cl. C12Q 1/68; G01N 33/574

(52) U.S. Cl. 435/6; 435/7.23

(57) ABSTRACT

A method of diagnosing predisposition to, or presence of ovarian cancer, breast cancer and/or lung cancer in a subject is provided. The method comprises determining a level of SIM2 in a lung tissue, breast tissue and/or ovarian tissue of the subject, the level being correlatable with predisposition to, or presence or absence of the ovarian cancer, breast cancer and/or lung cancer, thereby diagnosing predisposition to, or presence of ovarian cancer, breast cancer and/or lung cancer in the subject.

### FIELD AND BACKGROUND OF THE INVENTION

The present invention relates to SIM2 polypeptides and polynucleotides and to methods of diagnosing and treating ovarian, breast and lung cancers.

Lung cancer is the primary cause of cancer death among both men and women in the United States. The five-year survival rate among all lung cancer subjects, regardless of the stage of disease at diagnosis, is only 13%. This contrasts with a five-year survival rate of 46% among cases in which the disease is still localized. However, only 16% of lung cancers are diagnosed at a stage prior to spread of the disease.

Early detection is difficult since clinical symptoms are often not observed until the disease has reached an advanced stage. Currently, diagnosis is aided by the use of chest x-rays, analysis of the type of cells contained in sputum and fiberoptic examination of the bronchial passages. Treatment regimens are determined by the type and stage of the cancer, and include surgery, radiation therapy and/or chemotherapy. In spite of considerable research into therapies for the disease, lung cancer remains difficult to treat.

Breast cancer is the most common form of cancer in women, and can also be diagnosed in men. Over 200,000 new breast cancer cases are diagnosed each year in the United States. In the U.S. today, there are more than two million breast cancer survivors, and every woman is at risk.

Molecular biomarkers for breast cancer are of several types. Risk biomarkers are those associated with increased cancer risk and include mammographic abnormalities, proliferative breast disease with or without atypia, family clustering and inherited germ-line abnormalities. Surrogate endpoint biomarkers are tissue, cellular or molecular alterations that occur between cancer initiation and progression. These biomarkers are utilized as endpoints in short-term chemoprevention trials. Prognostic biomarkers provide information regarding outcome irrespective of therapy, while predictive biomarkers provide information regarding response to therapy. Candidate prognostic biomarkers for breast cancer include elevated proliferation indices such as Ki-67 and proliferating cell nuclear antigen (PCNA); estrogen receptor (ER) and progesterone receptor (PR) overexpression; markers of oncogene overexpression such as c-erbB-2, TGF-a and EGFR; indicators of apoptotic imbalance including overexpression of bcl-2 and an increased bax/bcl-2 ratio; markers of disordered cell signaling such as p53 nuclear protein accumulation; alteration of differentiation signals such as overexpression of c-myc and related proteins; loss of differentiation markers such as TGF-b II receptor and retinoic acid receptor; and alteration of angiogenesis proteins such as VEGF overexpression [Beenken (2002) Minerva Chir. 57:437-48].

Availability of molecular biomarkers for breast cancer will substantially increase diagnostic capabilities and enable the development of prognostic indices, which combine the predictive power of individual molecular biomarkers with specific clinical and pathologic factors.

Ovarian cancer, is a significant health problem for women in the United States and world wide. Although advances have been made in detection and therapy of this cancer, no vaccine or other universally successful method for prevention or treatment is currently available. Management of the disease currently relies on a combination of early diagnosis and aggressive treatment, which may include one or more of a variety of treatments such as surgery, radiotherapy, chemotherapy and hormone therapy. The course of treatment for a particular cancer is often selected based on a variety of prognostic parameters, including an analysis of specific tumor markers. However, the use of established markers often leads to a result that is difficult to interpret, and high mortality continues to be observed in many cancer subjects.

Thus, there remains a need for a practical method of diagnosing lung cancer, breast cancer and ovarian cancer as close to inception as possible. In order for early detection to be feasible, it is important that specific markers be found and their sequences elucidated.

The Drosophila single minded (sim) gene is the master regulator of fruit fly meurogenesis [Thomas (1988); Mambu (1991)]. SIM protein is a transcription factor containing a basic helix-loop-helix (bHLH) motif, two PAS (PER/ARNT/SIM) domains, and an HST (HIF-α/SIM/TRH) domain [Mambu (1991); Isaac and Andrew (1996)].

Two mouse homologs of the sim gene (i.e., Sim1 and Sim2) were cloned. Sim1 maps on mouse chromosome 10 and Sim2 on mouse chromosome 16 in a region of synteny with HC21 [Fan et al. (1996) Mol. Cell. Neurosci. 7:1-16]. Both mouse Sim1 and Sim2 genes are expressed early [Sim2 from embryonic day 8.0 (E8.0) and Sim1 from day 9.0 (E9.0)] in developing forebrain [Ema et al. (1996) Mol. Cell. Biol. 16:5865-5875; Fan et al. (1996) supra; Moffett et al. (1996) Genomics 35:144-155; Yamaki et al. (1996) Genomics 35:136-143] and outside the central nervous system (CNS), in somites, mesonephric duct, and foregut (SIM1), in facial and trunk cartilage, trunk muscles (Sim2), and in the developing kidney (SIM1 and SIM2) [Dahmane et al. (1995) Proc. Natl. Acad. Sci. 92:9191-9195; Ema et al. (1996) supra; Fan et al. (1996) supra; Moffett et al. (1996) supra].

In adult mouse, both SIM1 and SIM2 are expressed in kidney and skeletal muscles, whereas Sim2 is also expressed in the lung (Ema et al. (1996) Biochem. Biophys. Res. Commun. 218:588-594; Moffett et al. (1996) supra].

Recently, the cloning of the cDNAs for two human homologs (SIM1 and SIM2) of the Drosophila sim gene, and mapping of SIM1 to chromosome 6q16.3-q21 have been reported [Chrast (1997) Genome Research 7:615-624 and Chen et al. (1995)]. Northern blot analyses indicated the transcription of several mRNA transcripts from the SIM2 gene, including those of 2.7, 3, 4.4 and 6 kb. The multiple mRNAs may be products of alternative splicing, overlapping transcription, or different utilization of 5′ or 3′ untranslated sequences. At least two different forms of the human SIM2 gene have been characterized. The long form (GenBank Accession No. U80456; SEQ ID NO: 7 is 3921 bp and codes for a protein of 667 amino acid with an apparent molecular weight of 74 kD. The short-form (GenBank Accession No. U80457; SEQ ID NO: 8) is 2859 bp and codes for a protein of 570 amino acid with an apparent molecular weight of 64 kD.

Human SIM-1 and SIM-2, in combination with ARNT, attenuate transcription from the hypoxia-inducible erythropoietin (EPO) enhancer during hypoxia. SIM protein levels decrease with hypoxia treatment, suggesting a negative feedback mechanism. Upregulation and activation of HIF-1α is concomitant with attenuation of SIM activities [Woods S L., Whitelaw M L., J. Biol. Chem 2002; 277:10236-43].

PCT Application No. WO 02/12565 discloses the use of SIM2 as a marker and possible therapeutic target for specific types of cancer. Increased expression of SIM2 in specific cancers including colon, prostate and pancreas tumors as compared to normal tissues was observed. In accordance, a similar pattern of expression was found by DeYoung and co-worlers [Proc. Natl. Acad. Sci. (2003) 100:4760-5 and Anticancer Res. (2002) 22(6A):3149-57]. Interestingly, neither of the above-publications showed an elevated expression of SIM2 in ovarian, lung and breast tumors.

While reducing the present invention to practice the present inventors uncovered that elevated levels of SIM2 are associated with ovarian, breast and lung tumors, thus showing for the first time that SIM2 can also be used as a marker and possible therapeutic target for ovarian, breast and lung tumors.

### SUMMARY OF THE INVENTION

According to one aspect of the present invention there is provided a method of diagnosing predisposition to, or presence of ovarian cancer, breast cancer and/or lung cancer in a subject, the method comprising determining a level of SIM2 in a biological sample obtained from the subject, the level being correlatable with predisposition to, or presence or absence of the ovarian cancer, breast cancer and/or lung cancer, thereby diagnosing predisposition to, or presence of ovarian cancer, breast cancer and/or lung cancer in the subject.

According to further features in preferred embodiments of the invention described below, the biological sample is a tissue sample and/or a body fluid sample.

According to still further features in the described preferred embodiments the tissue sample is selected from the group consisting of an ovarian tissue, a lung tissue and a breast tissue.

According to another aspect of the present invention there is provided a method of treating ovarian cancer, breast cancer and/or lung cancer in a subject, the method comprising downregulating expression or activity of SIM2 in a lung tissue, breast tissue and/or an ovarian tissue, thereby treating the ovarian cancer, breast cancer and/or lung cancer in the subject.

According to still further features in the described preferred embodiments the SIM2 is selected from the group consisting of SEQ ID NOs: 1, 2, 3, 7, 8 and 9.

According to still further features in the described preferred embodiments downregulating expression or activity of the SIM2 is effected by administering to the subject:
(a) a molecule which binds SIM2;
(b) an enzyme which cleaves SIM2;
(c) an antisense polynucleotide capable of specifically hybridizing with an mRNA transcript encoding SIM2;
(d) a ribozyme which specifically cleaves SIM2 transcripts;
(e) a non-functional analogue of at least a catalytic or binding portion of SIM2;
(f) a molecule which prevents SIM2 activation or substrate binding;
(g) an siRNA molecule capable of inducing degradation of SIM2 transcripts;
(h) a DNAzyme which specifically cleaves SIM2 transcripts or DNA; and
(i) a molecule which promotes a SIM2-specific immunogenic response.

According to still further features in the described preferred embodiments the molecule which binds SIM2 is an antibody or antibody fragment capable of specifically binding the SIM2.

According to yet another aspect of the present invention there is provided use of an agent capable of downregulating SIM2 expression or activity for the treatment of ovarian, breast and/or lung cancer.

According to still further features in the described preferred embodiments the agent capable of downregulating SIM2 activity is an antibody or antibody fragment.

According to still further features in the described preferred embodiments the agent capable of downregulating SIM2 expression or activity is an oligonucleotide.

According to still further features in the described preferred embodiments the oligonucleotide is a single or double stranded polynucleotide.

According to still further features in the described preferred embodiments the oligonucleotide is at least 17 bases long.

According to still further features in the described preferred embodiments the oligonucleotide is hybridizable in either sense or antisense orientation.

According to still another aspect of the present invention there is provided use of a SIM2 detecting agent for detecting ovarian, breast and/or lung cancer.

According to still further features in the described preferred embodiments the agent for detecting ovarian, breast and/or lung cancer is an oligonucleotide.

According to still further features in the described preferred embodiments the agent for detecting ovarian, breast and/or lung cancer is an antibody or antibody fragment.

According to still further features in the described preferred embodiments the agent for detecting ovarian, breast and/or lung cancer is coupled to a detectable moiety selected from the group consisting of a chromogenic moiety, a fluorogenic moiety, a radioactive moiety and a light-emitting moiety.

According to an additional aspect of the present invention there is provided an article-of-manufacture comprising a packaging material and a composition identified for treating ovarian, breast and/or lung cancer being contained within the packaging material, the composition including, as an active ingredient, an agent capable of downregulating SIM2 expression or activity.

According to still an additional aspect of the present invention there is provided an isolated polynucleotide comprising a nucleic acid sequence encoding a polypeptide being at least 80% homologous to SEQ ID NO: 39, 46 or 41 as determined using the BestFit software of the Wisconsin sequence analysis package, utilizing the Smith and Waterman algorithm, where the gap creation equals 8 and gap extension penalty equals 2.

According to still further features in the described preferred embodiments the polypeptide is as set forth in SEQ ID NO: 39, 40 or 41.

According to a further aspect of the present invention there is provided an isolated polynucleotide comprising a nucleic acid sequence being 80% identical to SEQ ID NO: 39, 40 or 41, as determined using the BestFit software of the Wisconsin sequence analysis package, utilizing the Smith and Waterman algorithm, where gap weight equals 50, length weight equals 3, average match equals 10 and average mismatch equals −9.

According to still further features in the described preferred embodiments the nucleic acid sequence is as set forth in SEQ ID NO: 2 or 3.

According to yet a further aspect of the present invention there is provided an isolated polynucleotide as set forth in SEQ ID NO: 2 or 3.

According to still a further aspect of the present invention there is provided a nucleic acid construct comprising the isolated polynucleotide.

According to still a further aspect of the present invention there is provided an isolated polypeptide as set forth in SEQ ID NO: 39, 40 or 41.

According to still a further aspect of the present invention there is provided a method of diagnosing predisposition to, or presence of cancer in a subject, the method comprising determining a level of SEQ ID NO: 2 and/or 3 in a biological sample obtained from the subject, wherein the biological sample is suspected of being a cancerous tissue or associated with the cancerous tissue and whereas the level being correlatable with predisposition to, or presence or absence of the cancer, thereby diagnosing predisposition to, or presence of cancer in the subject.

According to still further features in the described preferred embodiments the determining level of the SEQ ID NO: 2 and/or 3 is effected at an mRNA level.

According to still further features in the described preferred embodiments the determining level of the SEQ ID NO: 2 and/or 3 is effected at a protein level.

According to still further features in the described preferred embodiments the determining level of the SEQ ID NO: 2 and/or 3 is effected at a gene amplification level.

According to still a further aspect of the present invention there is provided a method of treating cancer in a subject, the method comprising down-regulating expression or activity of SEQ ID NO: 2 and/or 3 in a cancerous tissue, thereby treating the cancer in the subject.

The present invention successfully addresses the shortcomings of the presently known configurations by providing SIM2 polypeptides and polynucleotides encoding same which can be used to diagnose and treat ovarian and lung cancers.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration showing the genomic organization of SIM2 expression products. Boxes designate exons and lines designate introns. Arrow heads designate primers.

FIG. 2 is a histogram showing SIM2 expression in normal and tumor-derived lung samples as determined by real time PCR using SEQ ID NO: 20, normalized to the housekeeping gene RPS27A (SEQ ID NO: 23). Two independent experiments are shown.

FIG. 3 is a histogram showing SIM2 expression in normal and tumor derived colon samples as determined by real time PCR using a SIM2 derived fragment (SEQ ID NO: 20).

FIG. 4 is a histogram showing SIM2 long and short transcript expression in normal and tumor-derived lung samples as determined by real time PCR using a SIM2 derived fragment (SEQ ID NO: 9) corresponding to coordinates 232-404 of SEQ ID Nos 7 and 8. Expression of SIM-2 derived fragment (SEQ ID NO: 9) was normalized to the expression of RPS27A (SEQ ID NO: 23) housekeeping gene. Two independent experiments are shown.

FIG. 5 is a histograms depicting SIM2 expression as in FIG. 4 on a 0-200 scale.

FIG. 6 is a histogram showing SIM2 long and short transcript (SEQ ID NOs: 7 and 8) expression in normal and tumor derived lung samples as determined by real time PCR using a SIM2 derived fragment (SEQ ID NO: 9). Expression of SEQ ID NO: 9 was normalized to the expression of RPS27A (SEQ ID NO: 23) housekeeping gene.

FIG. 7 is a histogram depicting SIM2 expression as in FIG. 6 on a 0-200 scale.

FIG. 8 is a histogram showing SIM2 expression in normal and tumor derived ovarian samples as determined by real time PCR using a SIM2 derived fragments (SEQ ID NOs: 9 and 20). Expression was normalized to the averaged expression of three housekeeping genes PBGD (SEQ ID NO: 32), ATP-6-syn (SEQ ID NO: 26) and 18s ribosomal RNA (SEQ ID NO: 29).

FIG. 9 is a histogram showing SIM2 long variant expression in normal and tumor derived ovary samples as determined by real time PCR using a SIM2 derived fragment (SEQ ID NO: 18). Expression was normalized to the averaged expression of two housekeeping genes PBGD (SEQ ID NO: 32) and HPRT1 (SEQ ID NO: 35).

FIG. 10 is a histogram showing SIM2 long variant expression in normal and tumor derived lung samples as determined by real time PCR using a SIM2 derived fragment (SEQ ID NO: 18). Expression was normalized to the averaged expression of three housekeeping genes SDHA (SEQ ID NO: 38), RPS27A (SEQ ID NO: 23) and PBGD (SEQ ID NO: 32).

FIG. 11 is a histogram showing SIM2 short variant expression in normal and tumor derived ovarian samples as determined by real time PCR using a SIM2 derived fragment (SEQ ID NO: 19). Expression was normalized to the averaged expression of two housekeeping genes PBGD (SEQ ID NO: 32) and HPRT1 (SEQ ID NO: 35).

FIG. 12 is a photomicrograph showing the expression of SIM2 long variant in normal and tumor derived breast samples as determined by RT-PCR using a SIM2 derived fragment (SEQ ID NO: 18).

FIG. 13 is a photomicrograph showing the expression of SIM2 short variant in normal and tumor derived breast samples as determined by RT-PCR using a SIM2 derived fragment (SEQ ID NO: 19).

### DESCRIPTION OF THE PREFERRED EMBODIMENTS

The present invention is of SIM2 polypeptides and polynucleotides encoding same, which can be used to diagnose and treat ovarian and lung cancers.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Lung cancer and ovarian cancer are deadly diseases for which treatment is currently limited. It is well established that early detection of primary, metastatic and recurrent diseases can significantly impact the prognosis of individuals suffering from lung cancer and ovarian cancer.

Current methods for early detection of ovarian cancer involve transabdominal/transvaginal ultrsonography and a blood test for the tumor marker CA-125. While ultrasound screening is often misleading since most enlarged ovaries result from benign cysts, the use of the tumor marker CA-125 in diagnosing ovarian cancer is limited due to a high false-positive rate.

Early detection of lung cancer is currently effected using chest X-rays and sputum cytology. Since these methods do not have any effect on mortality, detection still may be too late to affect the natural cause of the disease.

The Drosophila single minded (SIM) gene is the master regulator of fruit fly meurogenesis [Thomas (1988); Mambu (1991)]. SIM protein is a transcription factor containing a basic helix-loop-helix (bHLH) motif, two PAS (PER/ARNT/SIM) domains, and an HST (HIF-α/SIM/TRH) domain [Mambu (1991); Isaac and Andrew (1996)]. The native human SIM2 gene has been cloned and multiple mRNA products of the gene have been found by Northern blot analyses.

SIM2 has been previously associated with colon, pancreatic and prostate cancers but not with lung and ovarian cancers (PCT Application No. WO02/12565).

While reducing the present invention to practice the present inventors uncovered, for the first time, that elevated levels of SIM2 are present in ovarian, breast and lung tumors, thus providing evidence that this gene can be utilized as a diagnostic marker for ovarian, breast and lung tumors, or can serve as a basis for a therapeutic agent for treating such tumors.

Thus, according to one aspect of the present invention there is provided a method of diagnosing predisposition to, or presence of ovarian cancer, breast cancer and/or lung cancer in a subject.

As used herein the term “predisposition” refers to a latent susceptibility to ovarian, breast and/or lung cancer, which may lead, under certain conditions, to the formation of ovarian, breast and/or lung cancer.

As used herein the phrase “ovarian cancer” refers to epithelial tumors (i.e., carcinomas) and non-epithelial tumors (e.g., stroma cell and germ cell tumors of the ovary).

As used herein the phrase “breast cancer” refers to non-invasive and invasive tumors of the breast including Lobular Carcinoma In Situ (LCIS), Ductal carcinoma, Ductal Carcinoma In Situ (DCIS) and Carcinoma In Situ.

As used herein the phrase “lung cancer” refers to cancers of the lung including small cell lung cancer and non-small cell lung cancer.

The method, according to this aspect of the present invention is effected by determining a level of SIM2 in a biological sample which is obtained from the subject thereby diagnosing predisposition to, or presence of ovarian cancer, breast cancer and/or lung cancer in the subject.

As used herein “a biological sample” “refers to a sample of tissue (e.g., breast, lung, ovary) or fluid isolated from a subject, including but not limited to, for example, plasma, serum, spinal fluid, lymph fluid, the external sections of the skin, respiratory, intestinal, and genitourinary tracts, tears, saliva, milk, blood cells, tumors, organs, and also samples of in vivo cell culture constituents.

As used herein, the term “level” refers to expression of SIM2 RNA and/or protein or SIM2 DNA copy number.

As is further described hereinbelow and in the examples section which follows, the present inventors have shown that levels of SIM2 beyond those found in normal tissue correlate with predisposition to, or presence or absence of the ovarian cancer, breast cancer and/or lung cancer providing evidence that this gene can serve as a marker for breast cancer, lung cancer and ovarian cancer.

As used herein SIM2 refers to a SIM2 gene as set forth in sequence coordinates 34649835-34700062 of chromosome 21q22.13, expression products of the SIM2 gene as well as, fragments and variants thereof.

As used herein the term “variants” refers to splice variants and allelic variants of SIM2.

The phrase “splice variant” refers to alternative forms of RNA transcribed from a SIM2 gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a polypeptide encoded by a splice variant of an mRNA transcribed from a gene.

The phrase “allelic variant” refers to two or more alternative forms of a SIM2 gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

Examples of SIM2 expression products (i.e., splice variants) are set forth in SEQ ID NOs: 7 (GenBank Accession No. U80456) and 8 (GenBank Accession No. U80457), which correspond to the long form and short form of SIM2, respectively.

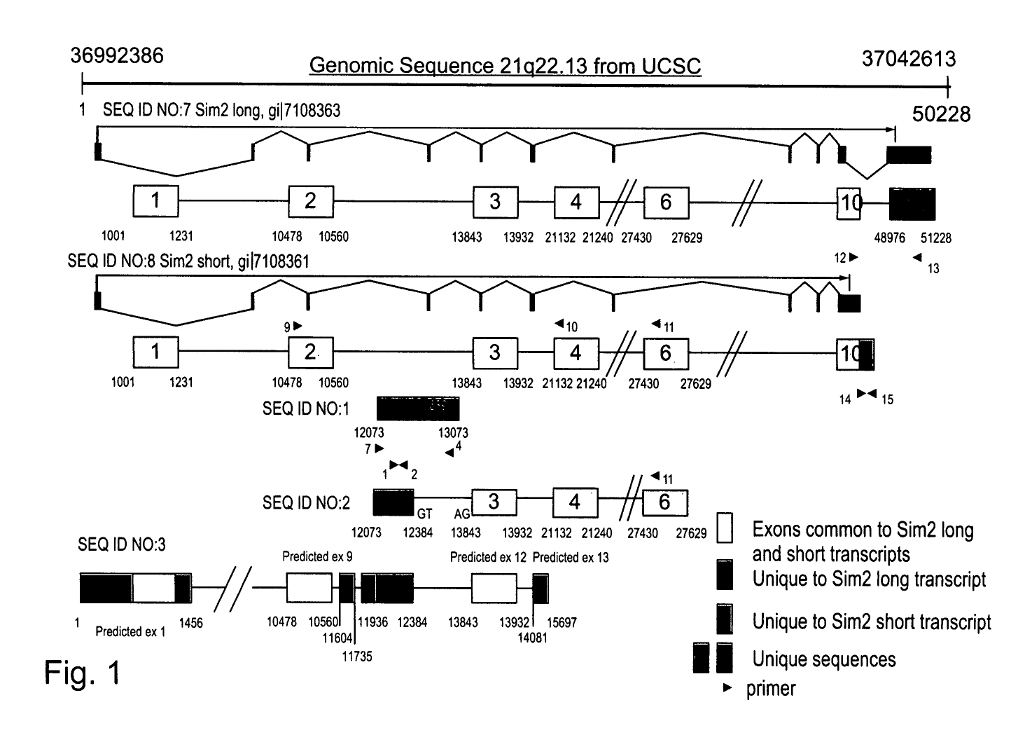

It will be appreciated that while reducing the present invention to practice additional SIM2 splice variant have been discovered by the present inventors. These are set forth in SEQ ID NOs: 2 and 3, further description of which is provided hereinbelow and in FIG. 1 and Table 1, below.

Numerous well known tissue or fluid collection (e.g., sputum collection) methods can be utilized to collect the biological sample from the subject in order to determine the level of SIM2 DNA, RNA and/or polypeptide of the subject.

Tissue biopsy can be utilized to collect a tissue sample from lung tissue, breast tissue and/or ovarian tissue. Methods of performing lung, breast and ovarian biopsies are well known in the art.

Typically, an ovarian biopsy is effected using fine-needle aspiration (FNA), which is an important diagnostic tool in gynecology. Its main role is in diagnosis of advanced and recurrent gynecologic malignancies. The technique uses a small-gauge needle to aspirate a lesion for cytologic analysis, sometimes with the aid of radiographic imaging.

A number of approaches for performing breast biopsies are known in the art.

A Stereotactic Needle Biopsy is a relatively novel approach, which is employed when the physician cannot feel the lump that was found on a mammogram. A Stereotactic Biopsy uses mammographic images and computer technology and combines them to determine the exact location of an abnormality to obtain a sample of breast tissue. In this way, non-surgical techniques are used to determine whether an abnormality is cancerous. This procedure is equally accurate as surgical biopsy without the scar and anesthetic risks of surgery. The patient lies facedown on a special table with an opening for the breast. Using special equipment, the breast is compressed similar to a mammogram but with less compression, and then x-rayed from several angles. The data is entered into a computer, and then a radiologist inserts a biopsy needle into the lump.

Fine Needle Aspiration involves inserting a very fine, hollow needle into a cyst to remove some fluid or tissue.

Core Needle Biopsy utilizes a larger needle inserted into the breast mass to remove a tissue for examination.

Ductal Lavage is a relatively new, minimally invasive FDA approved procedure being used for women who are considered at high risk of developing breast cancer. The doctor inserts a catheter into a milk duct and withdraws a sampling of cells.

Lung biopsies can be performed using a variety of techniques. A bronchoscopy is preferably effected to retrieve lung tissues which are located deep in the chest. If the area lies close to the chest wall, a needle biopsy is often done. If both these methods fail, an open surgical biopsy may be carried out. If there are indications that the lung cancer has spread to the lymph nodes in the mediastinum, a mediastinoscopy is performed.

When a needle biopsy is to be done, the subject will be given a sedative about an hour before the procedure, to help relaxation. The subject sits in a chair with arms folded on a table in front. X rays are then taken to identify the location of the suspicious areas. Small metal markers are placed on the overlying skin to mark the biopsy site. The skin is thoroughly cleansed with an antiseptic solution, and a local anesthetic is injected to numb the area.

A small cut (incision) about half an inch in length is then being made. The subject is asked to take a deep breath and hold it while a special biopsy needle is inserted through the incision into the lung. When enough tissue has been obtained, the needle is withdrawn. Pressure is applied at the biopsy site and a sterile bandage is placed over the cut. The entire procedure takes between 30 and 45 minutes.

The subject may feel a brief sharp pain or some pressure as the biopsy needle is inserted. Most subjects, however, do not experience severe pain.

Open biopsies are performed in a hospital under general anesthesia. As with needle biopsies, subjects are given sedatives before the procedure. An intravenous line is placed in the arm to give medications or fluids as necessary. A hollow endotracheal tube, is passed through the throat, into the airway leading to the lungs. It is used to convey the general anesthetic.

Once the subject is under anesthesia, an incision over the lung area is made. Some lung tissue is removed and the cut closed with stitches. The entire procedure takes about an hour. A chest tube is sometimes placed with one end inside the lung and the other end protruding through the closed incision. Chest tube placement is done to prevent the lungs from collapsing by removing the air from the lungs. The tube is removed a few days following the biopsy. A chest X ray is done following an open biopsy, to check for lung collapse.

The preparation for a mediastinoscopy is similar to that for an open biopsy. The subject is sedated and prepared for general anesthesia. The neck and the chest will be cleansed with an antiseptic solution. Once the subject is under anesthesia, an incision of about two or three inches long is made at the base of the neck. A thin, hollow, lighted mediastinoscope is inserted through the cut into the space between the right and the left lungs. The space is examined thoroughly and any lymph nodes or tissues that look abnormal are removed. The mediastinoscope is then removed, and the incision stitched up and bandaged.

Regardless of the procedure employed, once a biopsy is obtained the level of SIM2 can be determined and a diagnosis can thus be made.

Determining a level of SIM2 can be effected using various biochemical and molecular approaches used in the art for determining gene amplification, and/or level of gene expression.

It will be appreciated that since SIM2 is expressed in normal lung, breast and ovarian tissues at low levels, detection of SIM2 in a normal tissue is preferably effected along side to detect an elevated expression and/or amplification. Samples used to determine the normal range of SIM2 can be normal samples from individuals not suffering from the disease condition.

Typically, detection of a nucleic acid of interest in a biological sample is effected by hybridization-based assays using an oligonucleotide probe.

The term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly to respective naturally-occurring portions. An example of an oligonucleotide probe which can be utilized by the present invention is a single stranded polynucleotide which includes a sequence complementary to the sequence region encompassed by SEQ ID NO: 9 or 1.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The oligonucleotide of the present invention is of at least 17, at least 18, at least 19, at least 20, at least 22, at least 25, at least 30 or at least 40, bases specifically hybridizable with SIM2 derived sequence (e.g., SEQ ID NO: 1 which are hybridizable with the primers set forth in SEQ ID NOs. 4 and 5).

The oligonucleotides of the present invention may comprise heterocylic nucleosides consisting of purines and the pyrimidines bases, bonded in a 3′ to 5′ phosphodiester linkage.

Preferably used oligonucleotides are those modified in either backbone, internucleoside linkages or bases, as is broadly described hereinunder.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

It will be appreciated that olignoculeotides of the present invention may include further modifications which increase bioavailability, therapeutic efficacy and reduce cytotoxicity. Such modifications are described in Younes (2002) Current Pharmaceutical Design 8:1451-1466.

Hybridization based assays which allow the detection of SIM2 (i.e., DNA or RNA) in a biological sample rely on the use of oligonucleotide which can be 10, 15, 20, or 30 to 100 nucleotides long preferably from 10 to 50, more preferably from 40 to 50 nucleotides.

Hybridization of short nucleic acids (below 200 bp in length, e.g. 17-40 bp in length) can be effected using the following examplery hybridization protocols which can be modified according to the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ; (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample (target).

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, Fluor X (Amersham) and others [e.g., Kricka et al. (1992), Academic Press San Diego, Calif.] can be attached to the oligonucleotides.

Traditional hybridization assays include PCR, RT-PCR, RNase protection, in-situ hybridization, primer extension, Southern blot, Northern Blot and dot blot analysis.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

Specifically, gene amplification may be measured directly by DNA analysis such as Southern blot or dot blot techniques. For Southern blotting, DNA is extracted using methods which are well known in the art, involving tissue mincing, cell lysis, protein extraction and DNA precipitation using 2 to 3 volumes of 100% ethanol, rinsing in 70% ethanol, pelleting, drying and resuspension in water or any other suitable buffer (e.g., Tris-EDTA). Preferably, following such procedure, DNA concentration is determined such as by measuring the optical density (OD) of the sample at 260 nm (wherein 1 unit OD=50 μg/ml DNA).

To determine the presence of proteins in the DNA solution, the OD 260/OD 280 ratio is determined. Preferably, only DNA preparations having an OD 260/OD 280 ratio between 1.8 and 2 are used in the following procedures described hereinbelow.

The purified DNA is then digested with one or more restriction enzymes, and the resulting fragments separated on an agarose gel by electrophoresis. The DNA fragments are then transferred to a nylon or cellulose nitrate filter by blotting, and the DNA fixed by baking. The filter is then exposed to a labeled complementary probe and the regions of hybridization detected, usually by autoradiography. Dot blotting is similar, except that the DNA fragments are not separated on the gel. The degree of gene amplification is then determined by dilutional analysis or densitometry scanning.

Polymerase chain reaction (PCR)-based methods may be used to identify the presence of SIM2 mRNA. For PCR-based methods a pair of oligonucleotides is used, which is specifically hybridizable with the SIM2 polynucleotide sequences described hereinabove in an opposite orientation so as to direct exponential amplification of a portion thereof (including the hereinabove described sequence alteration) in a nucleic acid amplification reaction. For example, an oligonucleotide pair of primers specifically hybridizable with SIM2 are set forth in SEQ ID NOs: 10 and 11 which provide an amplification product which corresponds to SEQ ID NO: 9.

The polymerase chain reaction and other nucleic acid amplification reactions are well known in the art and require no further description herein. The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

Hybridization to oligonucleotide arrays may be also used to determine SIM2 expression. Such screening has been undertaken in the BRCA1 gene and in the protease gene of HIV-1 virus [see Hacia et al., (1996) Nat Genet 1996; 14(4):441-447; Shoemaker et al., (1996) Nat Genet 1996; 14(4):450-456; Kozal et al., (1996) Nat Med 1996; 2(7):753-759].

The nucleic acid sample which includes the candidate region to be analyzed is isolated, amplified and labeled with a reporter group. This reporter group can be a fluorescent group such as phycoerythrin. The labeled nucleic acid is then incubated with the probes immobilized on the chip using a fluidics station. For example, Manz et al. (1993) Adv in Chromatogr 1993; 33:1-66 describe the fabrication of fluidics devices and particularly microcapillary devices, in silicon and glass substrates.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Since the sequence and position of each probe immobilized on the chip is known, the identity of the nucleic acid hybridized to a given probe can be determined.

It will be appreciated that when utilized along with automated equipment, the above described detection methods can be used to screen multiple samples for ovarian, breast or lung cancers both rapidly and easily.

The presence of SIM2 in an ovarian, breast and/or lung tissue can also be determined at the protein level. Numerous protein detection assays are known in the art, examples include but are not limited to chromatography, electrophoresis, immunodetection assays such as ELISA and western blot analysis, immunohistochemistry and the like, which may be effected using antibodies specific to SIM2. Thus, the present invention envisages the use of serum immunoglobulins, polyclonal antibodies or fragments thereof, (i.e., immunoreactive derivatives thereof), or monoclonal antibodies or fragments thereof for the detection of ovarian, breast and/or lung cancer. Monoclonal antibodies or purified fragments of the monoclonal antibodies having at least a portion of an antigen-binding region, including the fragments described hereinbelow, chimeric or humanized antibodies and complementarily determining regions (CDR).

The term “antibody” refers to whole antibody molecules as well as functional fragments thereof, such as Fab, F(ab′)₂, and Fv that are capable of binding with antigenic portions of the target polypeptide. These functional antibody fragments constitute preferred embodiments of the present invention, and are defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule that can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“SCA”), a genetically engineered molecule containing the variable region of the light chain and the variable region of the heavy chain, linked by a suitable polypeptide linker as a genetically fused single chain molecule as described in, for example, U.S. Pat. No. 4,946,778.

SIM2-specific antibodies can be commercially obtained from Santa Cruz [SIM21 (C-17):sc-8716; SIM2s (C-15):sc-8715].

Alternatively, SIM2 specific antibodies may be generated using methods, which are well known in the art. See for example, Harlow and Lane, Antibodies: A Laboratory Manual, Cold Spring Harbor Laboratory, New York, 1988, incorporated herein by reference).

Purification of serum immunoglobulin antibodies (polyclonal antisera) or reactive portions thereof can be accomplished by a variety of methods known to those of skill including, precipitation by ammonium sulfate or sodium sulfate followed by dialysis against saline, ion exchange chromatography, affinity or immunoaffinity chromatography as well as gel filtration, zone electrophoresis, etc. (see Goding in, Monoclonal Antibodies: Principles and Practice, 2nd ed., pp. 104-126, 1986, Orlando, Fla., Academic Press). Under normal physiological conditions antibodies are found in plasma and other body fluids and in the membrane of certain cells and are produced by lymphocytes of the type denoted B cells or their functional equivalent. Antibodies of the IgG class are made up of four polypeptide chains linked together by disulfide bonds. The four chains of intact IgG molecules are two identical heavy chains referred to as H-chains and two identical light chains referred to as L-chains. Additional classes include IgD, IgE, IgA, IgM and related proteins.

Methods of generating and isolating monoclonal antibodies are well known in the art, as summarized for example in reviews such as Tramontano and Schloeder, Methods in Enzymology 178, 551-568, 1989. A recombinant SIM2 polypeptide may be used to generate antibodies in vitro. More preferably, the recombinant is used to elicit antibodies in vivo. In general, a suitable host animal is immunized with the recombinant SIM2. Advantageously, the animal host used is a mouse of an inbred strain. Animals are typically immunized with a mixture comprising a solution of the recombinant SIM2 in a physiologically acceptable vehicle, and any suitable adjuvant, which achieves an enhanced immune response to the immunogen. By way of example, the primary immunization conveniently may be accomplished with a mixture of a solution of the recombinant SIM2 and Freund's complete adjuvant, said mixture being prepared in the form of a water in oil emulsion. Typically the immunization will be administered to the animals intramuscularly, intradermally, subcutaneously, intraperitoneally, into the footpads, or by any appropriate route of administration. The immunization schedule of the immunogen may be adapted as required, but customarily involves several subsequent or secondary immunizations using a milder adjuvant such as Freund's incomplete adjuvant. Antibody titers and specificity of binding to the SIM2 can be determined during the immunization schedule by any convenient method including by way of example radioimmunoassay, or enzyme linked immunosorbant assay, which is known as the ELISA assay. When suitable antibody titers are achieved, antibody-producing lymphocytes from the immunized animals are obtained, and these are cultured, selected and cloned, as is known in the art. Typically, lymphocytes may be obtained in large numbers from the spleens of immunized animals, but they may also be retrieved from the circulation, the lymph nodes or other lymphoid organs. Lymphocytes are then fused with any suitable myeloma cell line, to yield hybridomas, as is well known in the art. Alternatively, lymphocytes may also be stimulated to grow in culture, and may be immortalized by methods known in the art including the exposure of these lymphocytes to a virus, a chemical or a nucleic acid such as an oncogene, according to established protocols. After fusion, the hybridomas are cultured under suitable culture conditions, for example in multi-well plates, and the culture supernatants are screened to identify cultures containing antibodies that recognize the hapten of choice. Hybridomas that secrete antibodies that recognize the recombinant SIM2 are cloned by limiting dilution and expanded, under appropriate culture conditions. Monoclonal antibodies are purified and characterized in terms of immunoglobulin type and binding affinity.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, in U.S. Pat. Nos. 4,036,945 and 4,331,647, and references contained therein, which patents are hereby incorporated by reference in their entirety (see also Porter, R. R., Biochem. J., 73: 119-126, 1959). Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of V_{H} and V_{L} chains. This association may be noncovalent, as described in Inbar et al. (Proc. Nat'l Acad. Sci. USA 69:2659-62, 1972). Alternatively, the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, all of which are hereby incorporated, by reference, in entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick and Fry Methods, 2: 106-10, 1991).

Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues, which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source, which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human monoclonal antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

Once the level of SIM2 is determined and the subject diagnosed, the diagnosis can be further validated using other diagnostic methods, which may also provide an accurate staging of the disease in the case of a positive diagnosis. Thus, for example, positive diagnosis for ovarian cancer may be confirmed by transabdominal/transvaginal ultrasonography and/or a blood test for the tumor marker CA-125. Ultrasound screening involves looking for enlarged ovaries and a transvaginal colour Doppler ultrasound is used to image blood flow. Blood vessel formation is thought to discriminate between cancer and benign cysts. Alternatively, positive diagnosis of lung cancer using the method of the present invention can be validated using chest X-rays and sputum cytology as well as spiral computed tomography (CT) scanning which can detect even very small tumors. Positive diagnosis of breast cancer using the method of the present invention can be validated using scintimammography, mammography, ultrasound and/or magnetic resonance imaging (MRI).

It will be appreciated that the above-described method of this aspect of the present invention may also be used to monitor disease progression and therapeutic regimen.

In addition to diagnostic advances pioneered by the present invention, the identification of overexpression of SIM2 in ovarian and lung cancers allows for the design of therapeutic agents, which can be used to treat ovarian, breast and lung cancers.

Thus, according to another aspect of the present invention there is provided a method of treating a subject (i.e., mammal e.g., human) having ovarian cancer, breast and/or lung cancer.

As used herein the term “treating” refers to preventing, curing, reversing, attenuating, alleviating, minimizing, suppressing or halting the deleterious effects of ovarian cancer and/or lung cancer.

The method according to this aspect of the present invention is effected by specifically downregulating expression or activity of SIM2 in a lung tissue, breast tissue and/or ovarian tissue to thereby treat the ovarian cancer, breast cancer and/or lung cancer in the subject.

Preferably, the method is effected by providing to the subject a therapeutically effective amount of an agent which is capable of downregulating SIM2 expression and/or activity.

As used herein “an agent capable of downregulating SIM2 expression and/or activity” refers to a molecule, which is capable of directly or indirectly down-regulating SIM2 expression or activity. An agent for direct downregulation of SIM2 refers to a molecule, which inhibits SIM2 intrinsic activity or expression. An agent for indirect downregulation of SIM2 refers to a molecule which inhibits the activity of a S[M2 effector (e.g., ARNT and HIF-1α) or expression thereof. The agents according to this aspect of the present invention can be a molecule which binds SIM2 (e.g., an antibody); an enzyme which cleaves SIM2; an antisense polynucleotide capable of specifically hybridizing with an mRNA transcript encoding SIM2; a SIM2 specific aptamer, a ribozyme which specifically cleaves SIM2 transcripts; a non-functional analogue of at least a catalytic or binding portion of SIM2 (e.g., a peptide-agent which can be identified using a phage display technology); a molecule which prevents SIM2 activation or substrate binding; an siRNA molecule capable of inducing degradation of SIM2 transcripts; a DNAzyme which specifically cleaves SIM2 transcripts or DNA, an activated double-stranded RNA (dsRNA-dependent protein kinase PKR as described in [Shir and Levitzki (2002) Nat. Biotechnol. 20(9):895-900 and Cell Mol Neurobiol. (2001) 21(6):645-56] and a molecule which is capable of promoting SIM2 specific immunization response.

One example, of an agent capable of downregulating a SIM2 is an antibody or antibody fragment capable of specifically binding SIM2 or an effector thereof. Examples of SIM2 antibodies are described hereinabove. Preferably, such antibodies are directed at functional domains of a target protein. Thus, for example, a SIM2 antibody according to this aspect of the present invention is preferably directed at the effector binding domain such as the ARNT binding domain. Alternatively, the antibody may bind a SIM2 effector such as ARNT or HIF-1α. An anti ARNT polyclonal rabbit serum raised against residues 1-140 of human ARNT and an anti HIF-1α polyclonal serum raised against residues 786-826 of human HIF-1α have been described by Susan (2002) J. Biol. Chem. 277:10236-10243. Preferably, the antibodies, according to this aspect of the present invention, are humanized such as described hereinabove.

Alternatively an agent capable of downregulating a SIM2 or an effector thereof can be a protease, which is designed to cleave SIM2. Proteases which can be used to cleave SIM2 can be identified by performing a computational analysis such as by using the SMART, MEME, MOTIFS, CDD-NCBI, BLOCKS or mPredict software each available from http://molbio.info.nih.gov/talks/tools/jobs.html and identifying a protease cleavage site.

Alternatively, agents which are designed to inhibit functional domains in the SIM2 protein (i.e., protein-protein interaction domains) can be computationally identified. For example, various peptide sequences derived from SIM2 can be computationally analyzed for an ability to bind an inhibitor using a variety of three dimensional computational tools. Software programs useful for displaying three-dimensional structural models, such as RIBBONS (Carson, M., 1997. Methods in Enzymology 277, 25), 0 (Jones, T A. et al., 1991. Acta Crystallogr. A47, 110), DINO (DINO: Visualizing Structural Biology (2001) http://www.dino3d.org); and QUANTA, INSIGHT, SYBYL, MACROMODE, ICM, MOLMOL, RASMOL and GRASP (reviewed in Kraulis, J., 1991. Appl Crystallogr. 24, 946) can be utilized to model interactions between SIM2 and prospective peptide and/or other small molecule inhibitors. Computational modeling of protein-peptide interactions has been successfully used in rational drug design, for further detail, see Lam et al., 1994. Science 263, 380; Wlodawer et al., 1993. Ann Rev Biochem. 62, 543; Appelt, 1993. Perspectives in Drug Discovery and Design 1, 23; Erickson, 1993. Perspectives in Drug Discovery and Design 1, 109, and Mauro M J. et al., 2002. J Clin Oncol. 20, 325-34. Specifically, the PRO_SELECT, tool for the virtual screening of libraries for fit to a protein active site, has been used to find novel leads against the serine protease factor Xa [Liebeschuetz J Med Chem. (2002);45(6):1221-32].

Another agent capable of downregulating a SIM2 or an effector thereof is a small interfering RNA (siRNA) molecule. RNA interference is a two-step process. the first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the SIM2 mRNA sequence of interest (e.g., SEQ ID NO: 7) is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. siRNA target sites on SEQ ID NO: 1 are preferably selected from the 5′ end i.e., coordinates 1-304 (see FIG. 1) e.g., nucleotide coordinates 171-193 of SEQ ID NO: 1. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Another agent capable of downregulating a SIM2 or an effector thereof is a DNAzyme molecule capable of specifically cleaving an mRNA transcript or DNA sequence of the SIM2. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides (e.g., nucleotide coordinates 311-326 of SEQ ID NO: 1), flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, LM [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al, 20002, Abstract 409, Ann Meeting Am Soc Gen Ther www.asgt.org). In another application, DNAzymes complementary to bcr-abl oncogenes were successful in inhibiting the oncogenes expression in leukemia cells, and lessening relapse rates in autologous bone marrow transplant in cases of CML and ALL.

Downregulation of a SIM2 or an effector thereof can also be effected by using an antisense polynucleotide capable of specifically hybridizing with an mRNA transcript encoding the SIM2 transcripts.

Design of antisense molecules, which can be used to efficiently downregulate a SIM2 must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof. An example antisense oligonucleotide which can be used in accordance with the present invention is designed to hybridize to nucleotide coordinates 311-400 of SEQ ID NO: 2. Such an antisense molecule hybridizes to a sequence which is shared by all SIM2 expression products known to date and as such may be useful in silencing SIM2 expression.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997); and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries

In addition, several approaches for designing and predicting efficiency of specific oligonucleotides using an in vitro system were also published (Matveeva et al., Nature Biotechnology 16: 1374-1375 (1998)].

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 (1999)].

More recently, antisense-mediated suppression of human heparanase gene expression has been reported to inhibit pleural dissemination of human cancer cells in a mouse model [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

It will be appreciated that antisense oligonucleotides can be used to modulate alternative splicing from SIM2 gene [Suzani and Kole (2003) Progress in Molecular and Subcellular Biology vol. 31 Philippe Jeanteur (Ed.) Springer-Verlag Berlin Heidelberg]. Inhibition of splicing by antisense oligonucleotides can be accomplished by targeting oligonucleotides to small nuclear RNAs (snRNAs), which participate in spliceosome formation and are essential for splicing and to splice sites and adjacent sequences [reviewed in Kole (1991) Adv. Drug Delivery Rev. 6:271-286].

Another agent capable of downregulating a SIM2 or an effector thereof is a ribozyme molecule capable of specifically cleaving an mRNA transcript encoding a SIM2 for example. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

Alternatively, the agent can be a molecule, which promotes a SIM2-specific immunogenic response in the subject. The molecule can be a SIM2 protein, a fragment derived therefrom or a nucleic acid sequence encoding thereof. Although such a molecule can be provided to the subject per se, the agent is preferably administered with an immunostimulant in an immunogenic composiiton. An immunostimulant may be any substance that enhances or potentiates an immune response (antibody and/or cell-mediated) to an exogenous antigen. Examples of immunostimulants include adjuvants, biodegradable microspheres (e.g., polylactic galactide) and liposomes into which the compound is incorporated (see e.g., U.S. Pat. No. 4,235,877). Vaccine preparation is generally described in, for example, M. F. Powell and M. J. Newman, eds., “Vaccine Design (the subunit and adjuvant approach),” Plenum Press (NY, 1995).

Illustrative immunogenic compositions may contain DNA encoding one or more of the SIM2 polypeptides as described above, such that the polypeptide is generated in situ. The DNA may be present within any of a variety of delivery systems known to those of ordinary skill in the art, including nucleic acid expression systems (see below), bacteria and viral expression systems. Numerous gene delivery techniques are well known in the art, such as those described by Rolland, Crit. Rev. Therap. Drug Carrier Systems 15:143-198, 1998, and references cited therein. Appropriate nucleic acid expression systems contain the necessary DNA sequences for expression in the subject (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

It will be appreciated that an immunogenic composition may comprise both a polynucleotide and a polypeptide component. Such immunogenic compositions may provide for an enhanced immune response.

Any of a variety of immunostimulants may be employed in the immunogenic compositions of this invention. For example, an adjuvant may be included. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2,-7, or -12, may also be used as adjuvants.

The adjuvant composition may be designed to induce an immune response predominantly of the Th1 type. High levels of Th1-type cytokines (e.g., IFN-.gamma., TNF.alpha., IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of an immunogenic composition as provided herein, the subject will support an immune response that includes Th1- and Th2-type responses. The levels of these cytokines may be readily assessed using standard assays. For a review of the families of cytokines, see Mosmann and Coffman, Ann. Rev. Immunol. 7:145-173, 1989.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corporation (Seattle, Wash.; see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant is a saponin, preferably QS21 (Aquila Biopharmaceuticals Inc., Framingham, Mass.), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210.

Other preferred adjuvants include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720.

A delivery vehicle may be employed within the immunogenic composition of the present invention to facilitate production of an antigen-specific immune response that targets tumor cells. Delivery vehicles include antigen presenting cells (APCs), such as dendritic cells, macrophages, B cells, monocytes and other cells that may be engineered to be efficient APCs. Such cells may be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Dendritic cells are highly potent APCs (Banchereau and Steinman, Nature 392:245-251, 1998) and have been shown to be effective as a physiological adjuvant for eliciting prophylactic or therapeutic antitumor immunity (see Timmernan and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naive T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within an immunogenic composition (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNF.alpha. to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNF.alpha., CD40 ligand, LPS, flt3 ligand and/or other compound(s) that induce differentiation, maturation and proliferation of dendritic cells.

Dendritic cells are categorized as “immature” and “mature” cells, which allows a simple way to discriminate between two well characterized phenotypes. Immature dendritic cells are characterized as APC with a high capacity for antigen uptake and processing, which correlates with the high expression of Fcy receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide encoding SIM2, such that SIM2, or an immunogenic portion thereof, is expressed on the cell surface. Such transfection may take place ex vivo, and a composition comprising such transfected cells may then be used for therapeutic purposes, as described herein. Alternatively, a gene delivery vehicle that targets a dendritic or other antigen presenting cell may be administered to the subject, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the SIM2 polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule) such as described above. Alternatively, a dendritic cell may be pulsed with a non-conjugated immunological partner, separately or in the presence of the polypeptide.

Agents for downregulating expression or activity of SIM2 (i.e., active ingredients) of the present invention can be provided to the subject per se, or as part of a pharmaceutical composition where they are mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the preparation accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Pharmaceutical compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

It will be appreciated that the oligonucleotides of the present invention can also be expressed from a nucleic acid construct, which can be administered to the subject employing any suitable mode of administration, described hereinabove (e.g., in-vivo gene therapy). Such a nucleic acid construct is introduced into a target cell or cells via appropriate gene delivery vehicle/methods (transfection, transduction, homologous recombination, etc.) and an expression system as needed and then the modified cells are expanded in culture and returned to the subject (i.e., ex-vivo gene therapy).

Such expression constructs may include a tissue-specific promoter for directing expression of the downregulating agents in the malignant tissue. Thus an ovarian specific promoter such as OSP-1 [Kumaran Cancer Res. (2001) February 15; 61(4):1291-5] and IAI.3B [Hamada Cancer Res. (2003) May 15; 63(10):2506-12] may be used. A breast-specific promoter which may be used in accordance with the present invention includes rNRL, Muc-1 and mWAP [Hiripi (2003) DNA Cell Biol. 22:41-5; Berger (2001) Breast Cancer Res. 3:28-35]. Alternatively a lung specific promoter such as CC-10 [Harrod Am J Respir Cell Mol. Biol. (2002) February; 26(2):216-23]and SP-C [Duan Oncogene. (2002) November 7; 21(51):7831-8]may be used Expression of duplex oligonucleotides is preferably effected via expression vectors specifically designed for such use. For example, the pSUPER™ including the polymerase-III H1-RNA gene promoter with a well defined start of transcription and a termination signal consisting of five thymidines in a row (T5) [Brummelkamp (2002) Science 296:550-53]. Another suitable siRNA expression vector encodes the sense and antisense siRNA under the regulation of separate polIII promoters [Miyagishi and Taira[(2002) Nature Biotech. 20:497-500]. The resultant siRNA includes 5 thymidine termination signal. Alternatively, oligonucleotide sequences can be placed under bi-directional promoters to produce both the sense and antisense transcripts from the same promoter construct, thus simplifying the construction of expression vectors and achieving an equal molar ratio of cellular sense and antisense sequences. Examples for bi-directional promoters are disclosed in U.S. Pat. Appl. No. 20020108142. It will be appreciated that when duplex oligonucleotide are used, transfection reagents dedicated to siRNA transfer to mammalian cells are preferably employed. Examples for such include but are not limited to siPORT™ Amine (i.e., a polyamine mixture) and siPORT™ Lipid (i.e., a mixture of cationic and neutral lipids).

Accordingly, in cases where the duplex oligonucleotides of the present invention are introduced into a cell in which RNA interference (RNAi) does not normally occur, the factors needed to mediate RNAi are introduced into such a cell or the expression of the needed factors is induced, as disclosed in U.S. Pat. Appl. No.: 20020086356.

It will be appreciated that treatment of subjects exhibiting mutated SIM2 transcripts may also be effected using a “knock in” strategy (see U.S. Pat. No. 6,265,632), wherein endogenous SIM2 sequence alterations are corrected using advanced gene therapy.

As is mentioned hereinabove, the present inventors uncovered novel isoforms of SIM-2.

Thus, according to another aspect of the present invention there is provided an isolated polynucleotide comprising a nucleic acid sequence encoding a polypeptide being at least 60%, at least 65%, at least 70%, at least 75%, at least 80%, at least 82%, at least 86%, at least 88%, at least 90%, at least 92%, at least 94% or more, say 95%-100% homologous to SEQ ID NO: 39, as determined using the BestFit software of the Wisconsin sequence analysis package, utilizing the Smith and Waterman algorithm, where the gap creation equals 8 and gap extension penalty equals 2.

According to one preferred embodiment of this aspect of the present invention the isolated polynucleotide is at least 60%, at least 65%, at least 70%, at least 75%, at least 80%, at least 82%, at least 86%, at least 88%, at least 90%, at least 92%, at least 94% or more, say 95%-100% identical to SEQ ID NO: 39, as determined using BestFit software of the Wisconsin sequence analysis package, utilizing the Smith and Waterman algorithm, where gap weight equals 50, length weight equals 3, average match equals 10 and average mismatch equals −9.

According to another preferred embodiment of this aspect of the present invention the isolated polynucleotide is as set forth in SEQ ID NO: 2.

According to yet another aspect of the present invention there is provided an isolated polynucleotide comprising a nucleic acid sequence encoding a polypeptide being at least 50%, at least 5%, at least 60%, at least 65%, at least 70%, at least 75%, at least 80%, at least 82%, at least 86%, at least 88%, at least 90%, at least 92%, at least 94% or more, say 95%-100% homologous to SEQ ID NOs: 40 or 41, as determined using the BestFit software of the Wisconsin sequence analysis package, utilizing the Smith and Waterman algorithm, where the gap creation equals 8 and gap extension penalty equals 2.

According to one preferred embodiment of this aspect of the present invention the isolated polynucleotide is at least 50%, at least 5%, at least 60%, at least 65%, at least 70%, at least 75%, at least 80%, at least 82%, at least 86%, at least 88%, at least 90%, at least 92%, at least 94% or more, say 95%-100% identical to SEQ ID NOs: 40 or 41, as determined using BestFit software of the Wisconsin sequence analysis package, utilizing the Smith and Waterman algorithm, where gap weight equals 50, length weight equals 3, average match equals 10 and average mismatch equals −9.

According to another preferred embodiment of this aspect of the present invention the isolated polynucleotide is as set forth in SEQ ID NO: 3.

As used herein the phrase “an isolated polynucleotide” refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Since the polynucleotide sequences of the present invention encode previously unidentified polypeptides, the present invention also encompasses isolated polypeptides or portions thereof which are encoded by the isolated polynucleotide which are described hereinabove.

Thus, this aspect of the present invention also encompasses polypeptides which are set forth in SEQ ID NO: 39, 40 or 41, homologues thereof (selected from the homology range of 60-100% described hereinabove) fragments thereof and altered polypeptides characterized by mutations, such as deletion, insertion or substitution of one or more amino acids, either naturally occurring or man induced, either randomly or in a targeted fashion.

Since expression of SIM2 is correlatable with cancer development (see WO 02/12565) the present invention also envisages the use of the novel sequences in diagnosis and treatment of cancer. Examples include, but are not limited to bone cancers, brain tumors, breast cancer, endocrine system cancers, gastrointestinal cancers, gynecological cancers, head and neck cancers, leukemia, lymphomas, metastases, myelomas, pediatric cancers, penile cancer, prostate cancer, sarcomas, skin cancers, thyroid cancer, thyoma, urinary tract cancers, carcinoma of unknown primary and Li-Fraumeni syndrome.

As is illustrated in the Examples section which follows, the present inventors have shown through laborious experimentation that these sequences are differentially expressed in colon adenocarcinoma, in lung adenocarcinoma and in lung squamous cell carcinoma supporting the use of such sequences in diagnosis and treatment of cancer as described above.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes 1-111 Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Genomic Organization of SIM2

Schematic presentation of SIM2 lung specific transcripts is shown in FIG. 1. Specifically, the genomic alignment of the exons of SEQ ID NOs: 1-3, 7 and 8 is shown. The genomic sequence used as a reference is chromosome 21q22.13, starting at position 36992386 and terminating at position 37042613. Genomic sequences are available at UCSC Genome Bioinformatics database, release version April 2002 (http:/genome.ucsc.edu).

Table 1, below shows the coordinates of the exons on the genomic sequence for each SEQ ID NO, correspondingly.

TABLE 1

| | | Relative exon |
|---|---|---|
| | Exon coordinates on | coordinates on |
| SEQ ID NO: | expressed sequence | genomic sequence |
| 1 | 1-1001 | 12073-13073 |
| 2 | 1-312 | 12073-12384 |
| 2 | 313-404 | 13843-13932 |
| 2 | 405-513 | 21132-21240 |
| 2 | 514-599 | 24356-24441 |
| 2 | 600-621 | 27430-27451 |
| 3 | 1-517 | 1001-1517 |
| 3 | 518-1766 | 1930-3178 |
| 3 | 1767-3143 | 5613-6989 |
| 3 | 3144-3473 | 7432-7761 |
| 3 | 3474-3640 | 8343-8509 |
| 3 | 3541-3895 | 9165-9419 |
| 3 | 3896-4159 | 9589-9852 |
| 3 | 4160-4644 | 10281-10765 |
| 3 | 4645-8296 | 11543-15194 |
| 7 | 1-231 | 1001-1231 |
| 7 | 232-314 | 10478-10560 |
| 7 | 315-404 | 13843-13932 |
| 7 | 405-513 | 21132-21240 |
| 7 | 514-599 | 24356-24441 |
| 7 | 600-799 | 27430-27629 |
| 7 | 800-906 | 32356-32462 |
| 7 | 907-1054 | 43028-43175 |
| 7 | 1055-1223 | 44698-44866 |
| 7 | 1224-1632 | 46039-46447 |
| 7 | 1633-3885 | 48976-51228 |
| 8 | 1-231 | 1001-1231 |
| 8 | 232-314 | 10478-10560 |
| 8 | 315-404 | 13843-13932 |
| 8 | 405-513 | 21132-21240 |
| 8 | 514-599 | 24356-24441 |
| 8 | 600-799 | 27430-27629 |
| 8 | 800-906 | 32356-32462 |
| 8 | 907-1054 | 43028-43175 |
| 8 | 1055-1223 | 44698-44866 |
| 8 | 1224-2823 | 46039-46447 |

### Example 2

### Materials and Exprerimental Procedures

RNA preparation—RNA was commercially obtained from Clontech (Franklin Lakes, N.J. USA 07417, www.clontech.com) or BioChain Inst. Inc. (www.biochain.com) or ABS or Clinomics. Alternatively RNA was purified from tissue samples using TR1-Reagent (Molecular Research Center), according to Manufacturer's instructions. Tissue samples were obtained from subjects or from postmortem. Total RNA samples were treated with DNaseI (Ambion) then purified using RNeasy columns (Qiagen).

Lung—Lu_{—}1N-Lu_{—}5N sample was prepared from commercially available normal lung RNAs (BioChain, Cat. No. CDP-061010, Lot Nos: A503205, A503384, A503385, A503204, A503206). RT Lu_{—}6N was prepared from a pool of 6 normal lung RNAs (BioChain, Cat. No. CDP-061010, Lot No. A409363).

Lu_{—}7C— Lu_{—}16C samples were prepared from lung adenocarcinoma RNAs: samples Lu_{—}7C— Lu_{—}10C were prepared from commercially available RNAs (BioChain, Cat. No. CDP-064004A, Lot Nos: A504117, A504119, A504116, A504118), and samples Lu_{—}11C-Lu_{—}16C were prepared from RNA purified from tissue samples of subjects.

RTs Lu_{—}17C-Lu_{—}31C were prepared from squamous cell carcinoma RNAs: samples Lu_{—}17C-Lu_{—}22C were prepared from commercial RNAs (BioChain, Cat. No. CDP-064004B, Lot Nos: A503187, A503386, A503387, A503183, A411075), sample Lu_{—}27C was prepared from commercial RNA (Clontech, Cat No: 64013-1), samples Lu_{—}28C-Lu_{—}30C were prepared from commercial RNAs (BioChain, Cat. No. CDP-064004, Lot Nos: A409017, A409091, A408175), and samples Lu_{—}23C-Lu_{—}26C and Lu_{—}31C were prepared from RNA purified from subjects tissue samples.

Lu_{—}32C-Lu_{—}35C were prepared from commercial small cell carcinoma RNAs (BioChain, Cat. No. CDP-064004D, Lot Nos: A504115, A501390, A501389, A501391).

Lu_{—}36C-Lu_{—}37C were prepared from commercial large cell carcinoma RNAs (BioChain, Cat. No. CDP-064004C, Lot Nos: A504113, A504114).

Lu_{—}38C was prepared from commercial alveolus cell carcinoma RNAs (BioChain, Cat. No. CDP-064004, Lot Nos: A409089).

RT Lu_{—}39C was prepared from lung carcinoma RNA purified from subject tissue sample (with no forther subcaracterization).

Lu_{—}40H1299 was prepared from RNA purified from NCI_H1299 cell line non-small cell carcinoma (ATCC Catalog No: CRL-5803).

SG_{—}41 was prepared from commercial normal salivary gland RNA (pool of 24) (Clontech, Cat No: 64110-1).

Lu_{—}16N and Lu_{—}25N were prepared from RNA purified from subjects normal tissue samples matched to the cancer samples Lu_{—}16C and Lu_{—}25C.

Colon—RTs marked as “Col_xN” (Col_{—}2N-Col_{—}276N) were prepared from normal colon RNAs. Samples Col_{—}22N and Col_{—}23N were prepared from commercial RNAs (BioChain, Cat. No. CDP-064007, Lot Nos: A501132, A501130), and the rest were prepared from RNA purified from normal subjects tissue samples. Normal pool sample was prepared form comercial RNA pool of normal colon (BioChain, Cat. No. CDP-061003, Lot Nos: A411078).

RTs Col_{—}2C-Col_{—}23C were prepared from colon adenocarcinoma RNAs, matched to the normal samples Col_{—}2N-Col_{—}23N. Samples Col_{—}22C and Col_{—}23C were prepared from commercial RNAs BioChain, Cat. No. CDP-064007, Lot Nos: A501131, A501129), and the rest were prepared from RNA purified from subjects colon adenocarcinoma cancer tissue samples.

Colon cell lines—Col-SW620 is epithelial colorectal adenocarcinoma from metastatic lymph node, Dukes C (ATCC Catalog No: CCL-227), Col-SW480 is epithelial colorectal adenocarcinoma, Dukes B (ATCC Catalog No: CCL-228) and Col-DLD1 is epithelial colorectal adenocarcinoma, Dukes C (ATCC Catalog No: CCL-221).

Ovary—Ovarian RNA was generated as described in Table 2, below.

TABLE 2

| Serial number | Lot number | Source | Tissue | Pathology (grade) |
|---|---|---|---|---|
| 1-Pap Adeno G3 | ILS-1406 | ABS | ovary | papillary adenocarcinoma (3) |
| 2-Pap Adeno G2 | ILS-1408 | ABS | ovary | papillary adenocarcinoma (2) |
| 3-Pap Adeno G2 | ILS-1431 | ABS | ovary | papillary adenocarcinoma (2) |
| 4-Pap CystAde G2 | ILS-7286 | ABS | ovary | papillary cystadenocarcinoma (2) |
| 5-Adeno G3 | 99-12-G432 | GOG | ovary | adenocarcinoma (3) |
| 6-Adeno G3 | A0106 | ABS | ovary | adenocarcinoma (3) |
| 7-Adeno G3 | IND-00375 | ABS | ovary | adenocarcinoma (3) |
| 8-Adeno G3 | A501113 | BioChain | ovary | adenocarcinoma (3) |
| 9-Adeno G3 | 99-06-G901 | GOG | ovary | adenocarcinoma (maybe serous) |
| | | | | (3) |
| 10-Adeno G3 | A407069 | Biochain | ovary | adenocarcinoma (3) |
| 11-Adeno G3 | A407068 | Biochain | ovary | adenocarcinoma (3) |
| 12-Adeno G3 | A406023 | Biochain | ovary | adenocarcinoma (3) |
| 13-Adeno G3 | 94-05-7603 | GOG | right ovary | metastasis adenocarcinoma (3) |
| 14-Adeno G2 | A501111 | BioChain | ovary | adenocarcinoma (2) |
| 15-Carcinoma G3 | A407065 | BioChain | ovary | carcinoma (3) |
| 16-Carcinoma | 1090387 | Clontech | ovary | carcinoma NOS |
| 17-Muc Adeno G3 | A504084 | BioChain | ovary | mucinous adenocarcinoma (3) |
| 18-Muc Adeno G3 | A504083 | BioChain | ovary | mucinous adenocarcinoma (3) |
| 19-Muc Adeno G3 | A504085 | BioChain | ovary | mucinous adenocarcinoma |
| 20-Pap Muc CystAde | USA-00273 | ABS | ovary | papillary mucinous |
| | | | | cystadenocarcinoma |
| 21-Muc CystAde G2-3 | 95-10-G020 | GOG | ovary | mucinous cystadenocarcinoma (2- |
| | | | | 3) |
| 22-Muc CystAde G2 | A0139 | ABS | ovary | mucinous cystadenocarcinoma (2) |
| 23-Muc CystAde G3 | VNM- | ABS | ovary | mucinous cystadenocarcinoma |
| | 00187 | | | with low malignant (3) |
| 24-Pap Sero Adeno G3 | 2001-07- | GOG | ovary | papillary serous adenocarcinoma |
| | G801 | | | (3) |
| 25-Pap Sero Adeno G3C | N0021 | ABS | ovary | papillary serous adenocarcinoma |
| | | | | (3C) |
| 26-Sero Adeno G3 | 2001-12- | GOG | right ovary | serous adenocarcinoma (3) |
| | G035 | | | |
| 27-Pap Sero Carci G3 | 2001-08- | GOG | ovary | papillary serous carcinoma (3) |
| | G011 | | | |
| 28-Pap Sero CystAde G3 | A503176 | BioChain | ovary | serous papillary |
| | | | | cystadenocarcinoma (3) |
| 29-Pap Sero CystAde G3 | 93-09-4901 | GOG | ovary | serous papillary |
| | | | | cystadenocarcinoma (3) |
| 30-Pap Sero CystAde G3 | A503175 | BioChain | ovary | serous papillary |
| | | | | cystadenocarcinoma (1) |
| 31-Pap Endo Adeno G3C | 95-04-2002 | GOG | ovary | papillary endometrioid |
| | | | | adenocarcinoma (3C) |
| 32-Endo Adeno G2 | 94-08-7604 | GOG | right ovary | endometrioid adenocarcinoma (2) |
| 33-Endo Adeno G1-2 | 2000-09- | GOG | ovary | endometrial adenocarcinoma (1-2) |
| | G621 | | | |
| 34-Mix Sero/Endo G3 | 2002-05- | GOG | ovary | mixed serous and endometrioid |
| | G513 | | | adenocarcinoma (3) |
| 35-Mix Sero/Endo G3 | 2002-05- | GOG | ovary | mixed serous and endometrioid |
| | G509 | | | adenocarcinoma of mullerian (3) |
| 36-Mix Sero/Endo G3 | 2001-12- | GOG | ovary | mixed serous and endometrioid |
| | G037 | | | adenocarcinoma (3) |
| 37-Mix Sero/Endo G2 | 95-11-G006 | GOG | ovary, | papillary serous and endometrioid |
| | | | endometrium | cystadenocarcinoma (2) |
| 38-Mix Sero/Muc/Endo G2 | 98-03-G803 | GOG | ovary | mixed epithelial |
| | | | | cystadenocarcinoma with |
| | | | | mucinous, endometrioid, |
| | | | | squamous and papillary serous(2) |
| 39-Adeno borderline | 98-08-G001 | GOG | ovary | epithelial adenocarcinoma of |
| | | | | borderline malignancy |
| 40-Clear cell Adeno G3 | 2001-10- | GOG | ovary | clear cell adenocarcinoma (3) |
| | G002 | | | |
| 41-Clear cell Adeno | 2001-07- | GOG | ovary | clear cell adenocarcinoma |
| | G084 | | | |
| 42-N | A503274 | BioChain | ovary | Normal |
| 43-N | A504086 | BioChain | ovary | Normal |
| 44-N | 061P43A | Ambion | ovary | Normal |
| 45-N | A504087 | BioChain | ovary | Normal |
| 46-N M14 | A501112 | BioChain | ovary | Normal (matched tumor A501111) |
| 47-N M8 | A501114 | BioChain | ovary | Normal (matched tumor A501113) |
| 48-N M38 | 98-03- | GOG | ovary | Normal (matched tumor 98-03- |
| | G803N | | | G803) |
| 49-N M39 | 98-08- | GOG | ovary | Normal (matched tumor 98-08- |
| | G001N | | | G001) |

RT reaction with oligo-dT—Reverse transcription was effected using 2 μg of total RNA, in a 20 μl reaction, including 200 units of Superscript II Reverse Transcriptase (Bibco/BRL) in the buffer supplied by the manufacturer, 500 pmol of oligo(dT)₂₅ (Promega Corp. Madison Wis., USA), and 40 units of RNasin (Promega Corp. Madison Wis., USA).

Real Time PCR—5 ul RT reaction products, diluted in final reaction volume of 20 ul was used for amplification. Specific oligonucleotides (SEQ ID NO:4 and SEQ ID NO:5) were used as primers. The ABI Prism 7000 Sequence Detection System was used for cycling. The reaction was effected using SYBR GreenPCR Master Mix (Applied Biosystems). The cycle in which the reactions achieved a threshold level (Ct) of fluorescence was registered and served to calculate the initial transcript quantity in the RT reaction. Control PCR reactions were effected on the same RT sample, using PCR primers specific to the house keeping gene ribosomal protein S27a (RPS27A). An amplicon fragment thereof (SEQ ID NO: 23) was generated using the primers set forth in SEQ ID NOs: 21 and 22. For each primer set, the value of each PCR reaction was divided into the value of one of the RTs (a pool of normal lung transcripts from BioChain). In order to normalize the results, the ratio for each PCR reaction was then divided by the house keeping gene ratio for the same RT.

RT PCR for Examples 6-9—1 μg of treated RNA was mixed with 150 ng Random Hexamer primers (Invitrogen) and 500 μM dNTP in total volume of 15.6 μl. The mixture was incubated for 5 min at 65° C. and then quickly chilled on ice. Then, 5 μl 5× SuperscriptII first strand buffer (Invitrogen), 2.4 μl 0.1 M DTT and 40 units Rnasin (Promega) were added, and the mixture was incubated for 10 min at 25° C., followed by further incubation at 42° C. for 2 min. Then, 1 μl (200 units) of SuperscriptII (Invitrogen) was added and the reaction (final volume of 25 μl) was incubated for 50 min at 42° C. and then inactivated at 70° C. for 15 min. The resulting cDNA was diluted 1:20 in 10 mM T0.1E.

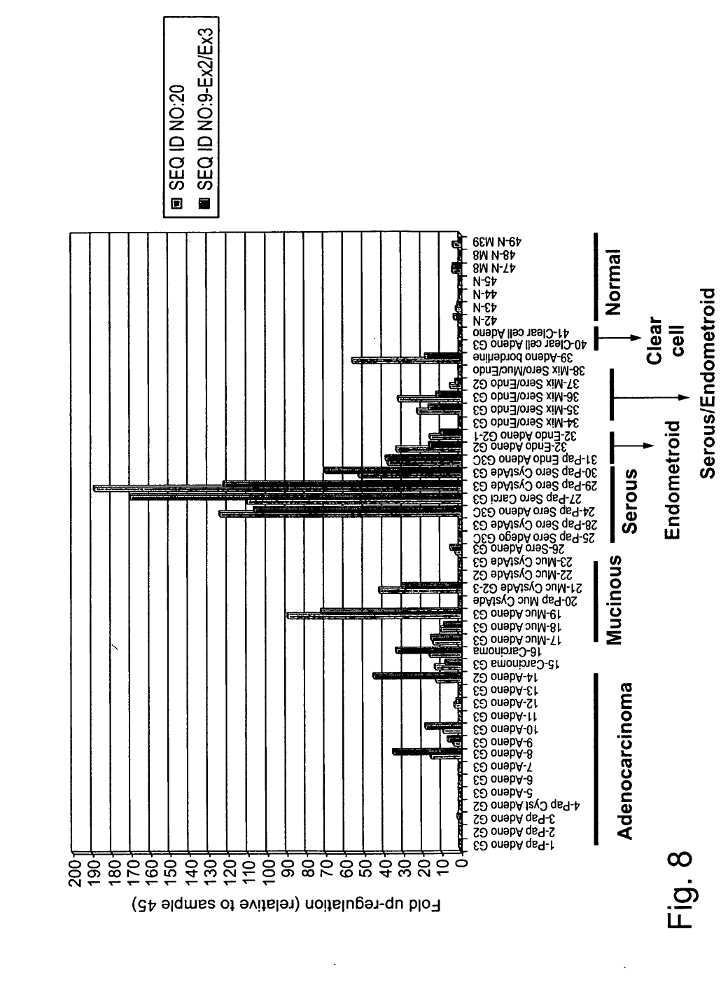

Real-Time RT-PCR used for analyzing expression pattern of SEQ ID NOs. 1, 9, 18 and 19 in different ovary and lung samples (Examples 7-9, FIGS. 8-11)-5 μl of diluted cDNA prepared with random primers were used as a template in Real-Time PCR reactions using the SYBR Green I assay (PE Applied Biosystem) with specific primers. The amplification stage was effected as follows, 50° C. for 2 min, 95° C. for 10 min, 95° C. for 15 sec, followed by 60° C. for 1 min. Detection was effected using PE Applied Biosystem SDS 7000. The cycle in which the reactions achieved a threshold level (Ct) of fluorescence was registered and served to calculate the initial transcript quantity in the RT reaction. The quantity was calculated using a standard curve created using serial dilutions of either a purified amplicon product or reverse transcription (RT) reaction prepared from RNA mix purified from 5 cell-lines (HCT116, H1299, DU145, MCF7, ES-2). To minimize inherent differences in the RT reaction, the resulting quantity was normalized to the geometric mean of the quantities of several housekeeping genes (different HSKP genes were used for the different tissue panels).

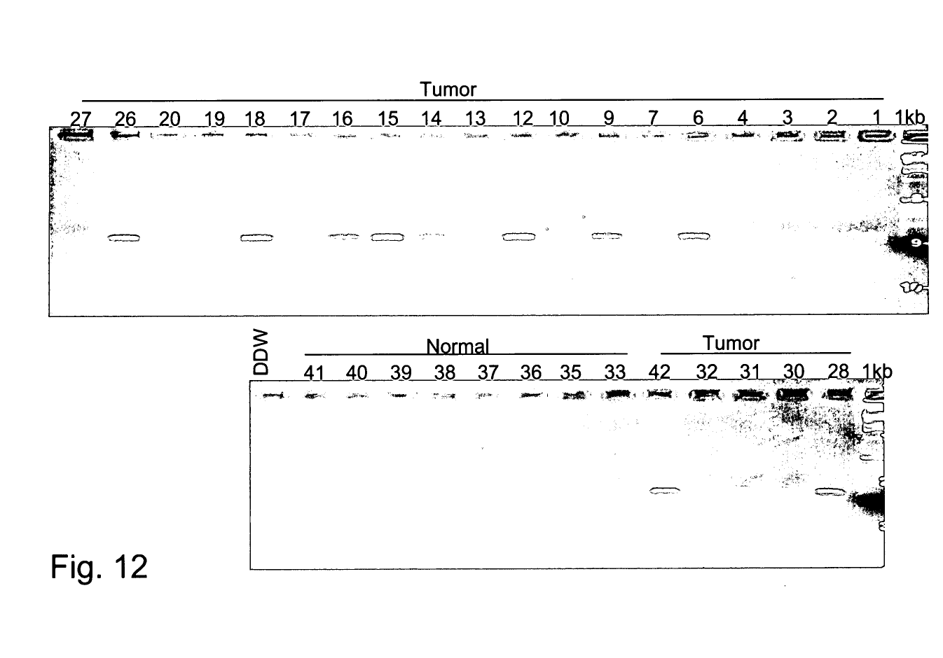

RT-PCR analysis used for analyzing expression pattern of SEQ ID NOs. 18 and 19 in different breast samples (examples 10, 11, FIGS. 12,13)-5 μl of diluted cDNA prepared with random primers were used as a template in RT-PCR reactions using the SYBR Green I assay (PE Applied Biosystem) with specific primers. The amplification stage was effected as follows, 50° C. for 2 min, 95° C. for 10 min, 95° C. for 15 sec, followed by 60° C. for 1 min. PCR products were analyzed by 1.8% agarose gels.

### Example 3

### Expression Pattern of a SIM2 Derived Fragment (SEQ ID NO:20) in Normal and Malignant Lung Samples

The expression level of a SIM2 derived fragment corresponding to SEQ ID NO:20, which is a fragment of SEQ ID NO: 1 (nucleotide coordinates 125-225, see FIG. 1) was determined using the primers set forth in SEQ ID NOs: 4 and 5 (designated as primers 1 and 2 in FIG. 1) and measured by real time PCR. The expression of the housekeeping gene RPS27A (GenBank Accession No. NM_{—}002954, SEQ ID NO: 23) was determined similarly using primers: SEQ ID NOs: 21, 22. Expression values were first normalized to a housekeeping gene. The expression was then calculated relative to a pool of normal lung samples (Lu_{—}6N).

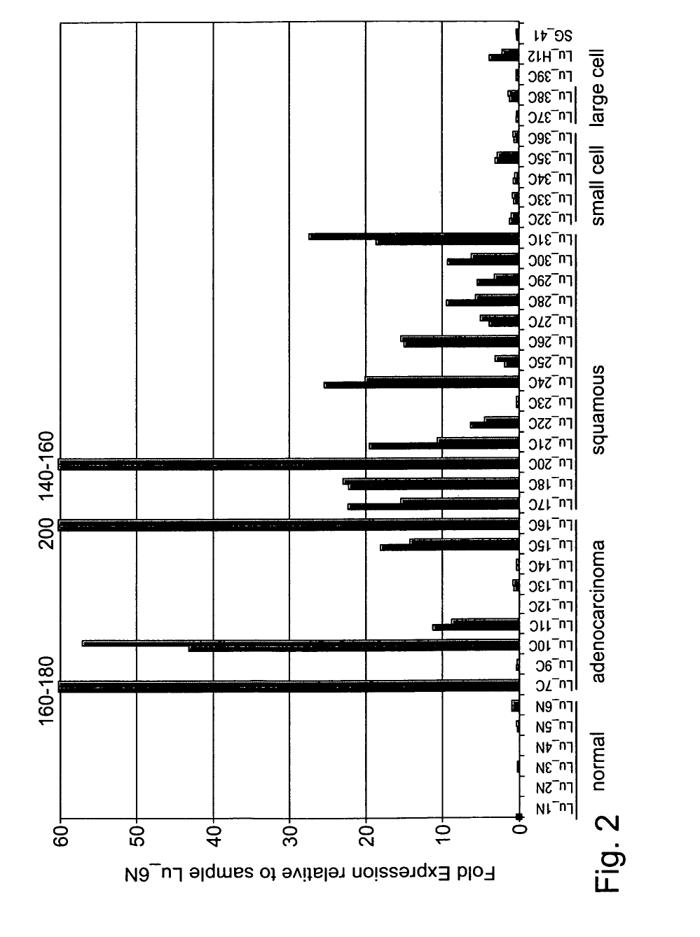

As shown in FIG. 2, representing two duplicates of the same experiment, the expression of the SIM2 fragment (SEQ ID NO: 1) in the normal samples was significantly lower than in the tumor samples. Interestingly, high expression was found in adenocarcinoma samples (4 out of the 9 samples) and squamous cell carcinoma samples (7 out of the 14 samples). The expression in these samples was between 10 and 200 fold higher as compared to the expression pattern in normal samples.

### Example 4

### Expression Pattern of a SIM2 Derived Fragment (SEQ ID NO:20) in Normal and Malignant Colon Samples

The expression levels of a SIM2 derived fragment corresponding to SEQ ID NO. 20 (a portion of SEQ ID NO: 1 amplified by the primers set forth in SEQ ID NOs. 4-5) as well as the housekeeping gene RPS27A (GenBank Accession No. NM_{—}002954, SEQ ID NO: 23) using primers: SEQ ID NOs: 21, 22) were measured by real time PCR. Each value was first normalized to the housekeeping gene. The expression level was then calculated relative to a pool of RNA from normal colon (Col-normal pool).

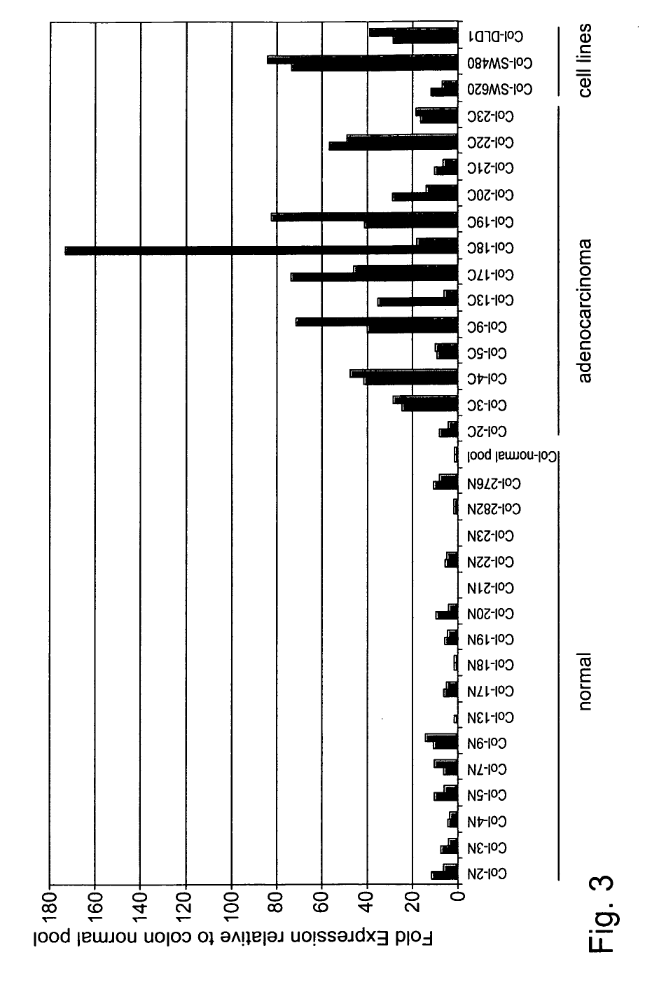

As shown in FIG. 3, representing two duplicates of the same experiment, the expression in most of the adenocarcinoma and cell lines samples was higher than in the normal samples.

### Example 5

### Expression Pattern of a SIM2 Derived Fragment (SEQ ID NO: 9) in Normal and Malignant Lung Samples

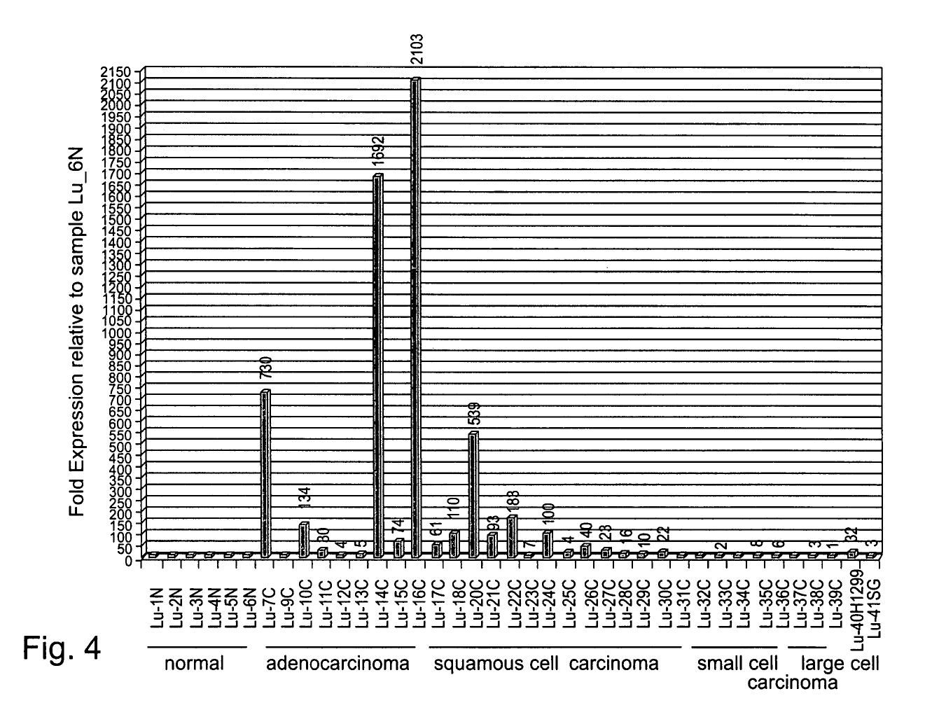

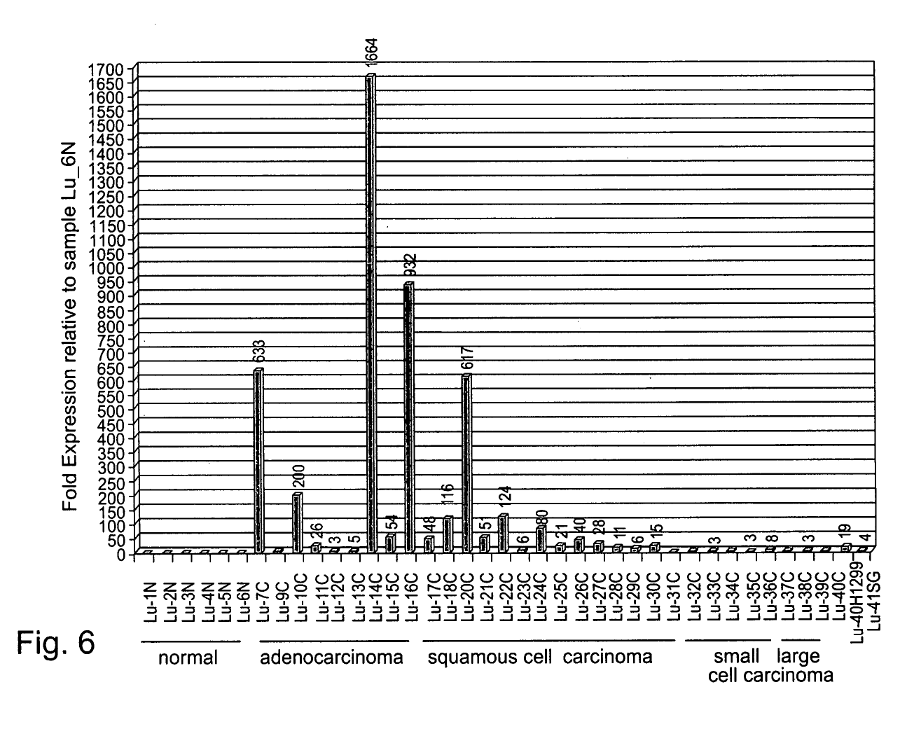

Expressions of long (SEQ ID NO: 7) and short (SEQ ID NO: 8) SIM variants was measured by real time PCR using a sequence fragment (SEQ ID NO: 9, and the primers set forth in SEQ ID NOs: 10 and 11, designated as primers 9 and 10 in FIG. 1, respetively) which is shared by both sequences. Each expression was first normalized to RPS27A (SEQ ID NO: 23; using primers: SEQ ID NOs: 21, 22) (FIGS. 4 through 7). Then the expression level was calculated relative to a pool of normal lungs (Lu_{—}6N).

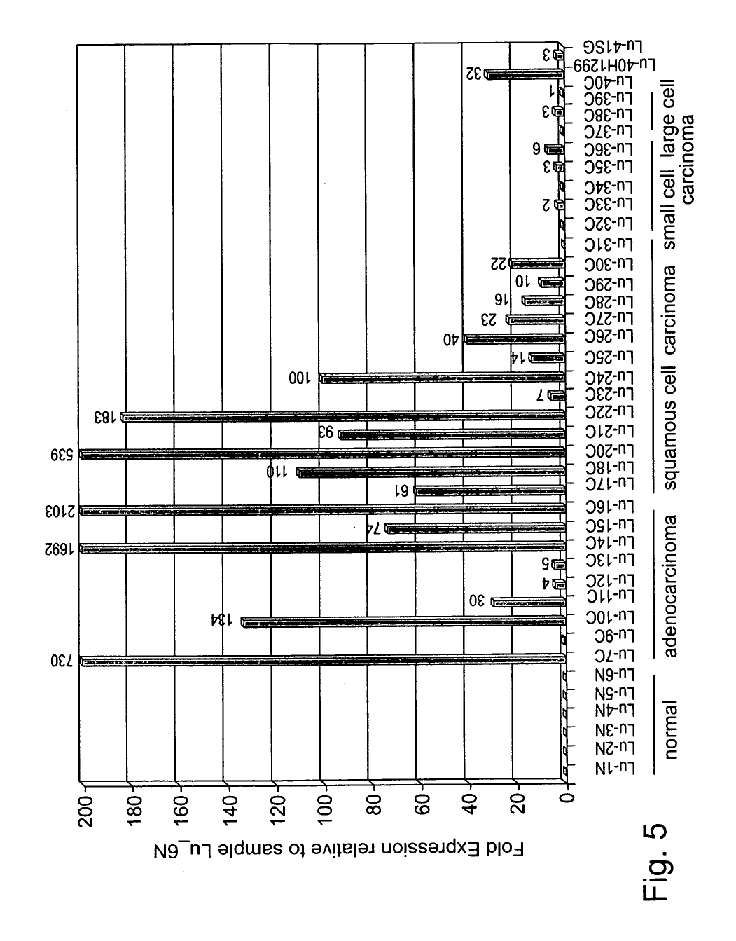

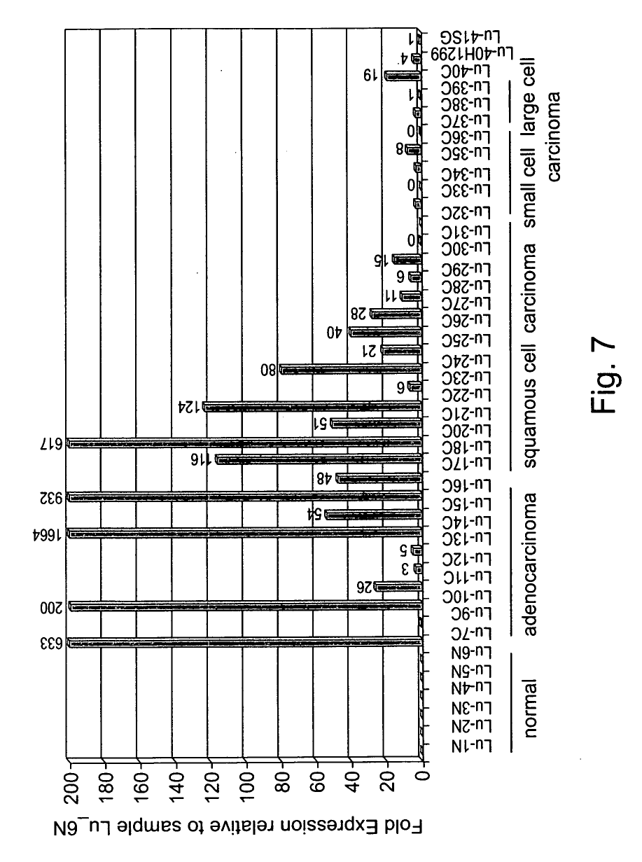

As shown in FIGS. 4 through 7, SIM2 expression in normal samples was very low. High expression was found in adenocarcinoma samples and squamous cell carcinoma samples. Note that over-expression in tumor samples was 10 to 2000 fold relatively to the expression of SIM2 in normal samples. FIGS. 5 and 7 show a better resolution pattern of SIM2 expression on a scale of 0-200.

### Example 6

### Expression of SIM2 Derived Fragments in Normal and Cancerous Ovary Tissues

The expression of two different SIM2-derived sequences (SEQ ID NOs:20 and 9) and three housekeeping genes—PBGD (GenBank Accession No: HSPBGDR, SEQ ID NO: 32; using the primers set forth in SEQ ID NOs: 30, 31), ATP-6-syn (GenBank Accession No: NM_{—}1733702, SEQ ID NO: 26; using the primers set forth in SEQ ID NOs: 24, 25), 18s ribosomal RNA (GenBank Accession No: HSRRN18S, SEQ ID NO: 29; using the primers set forth in SEQ ID NOs: 27, 28) were measured by real time PCR. In each RT sample, the expression of SIM2 sequences was normalized to the geometric mean of the quantities of three housekeeping genes PBGD, ATP-6-syn, 18s ribosomal RNA, as detailed in Example 2, hereinabove. The normalized quantity of each RT sample was then divided by the normalized quantity of a normal sample (No. 45, Table 2 above).

As shown in FIG. 8, SIM2 expression in normal samples (samples nos. 42-49, Table 2 above) was significantly lower than in the cancer samples. Notably, the highest expression of SIM2 was found in papilary serous (carcinoma or adenocarcinoma or cystadenocarcinoma) samples (samples 24, 27, 29, Table 2).

### Example 7

### Expression of SIM2 Long Variant-Derived Fragment (SEQ ID NO:18) in Normal and Cancerous Ovary Tissues

Expression of SIM2 long variant (GenBank Accession No: gi7108363, SEQ ID NO: 7) was measured by real time PCR using a fragment (SEQ ID NO: 18 corresponding to nucleotide coordinates 1551-1670 of SEQ ID NO: 7 using the primers set forth in SEQ ID NOs:14-15, designated as primers 12 and 13 in FIG. 1, respectively). In addition the expression of two housekeeping genes—PBGD (GenBank Accession No: HSPBGDR, SEQ ID NO: 32; using the primers set forth in SEQ ID NOs: 30-31) and HPRT1 (GenBank Accession No: GI_{—}32449, SEQ ID NO: 35; using the primers set forth SEQ ID NOs: 33-34), was measured by real time PCR. In each RT sample, the expression of SIM2 sequences was normalized to the geometric mean of the quantities of the housekeeping genes as detailed in Example 2, hereinabove. The normalized quantity of each RT sample was then divided by the averaged quantity of the normal samples (No. 42-48, Table 2).

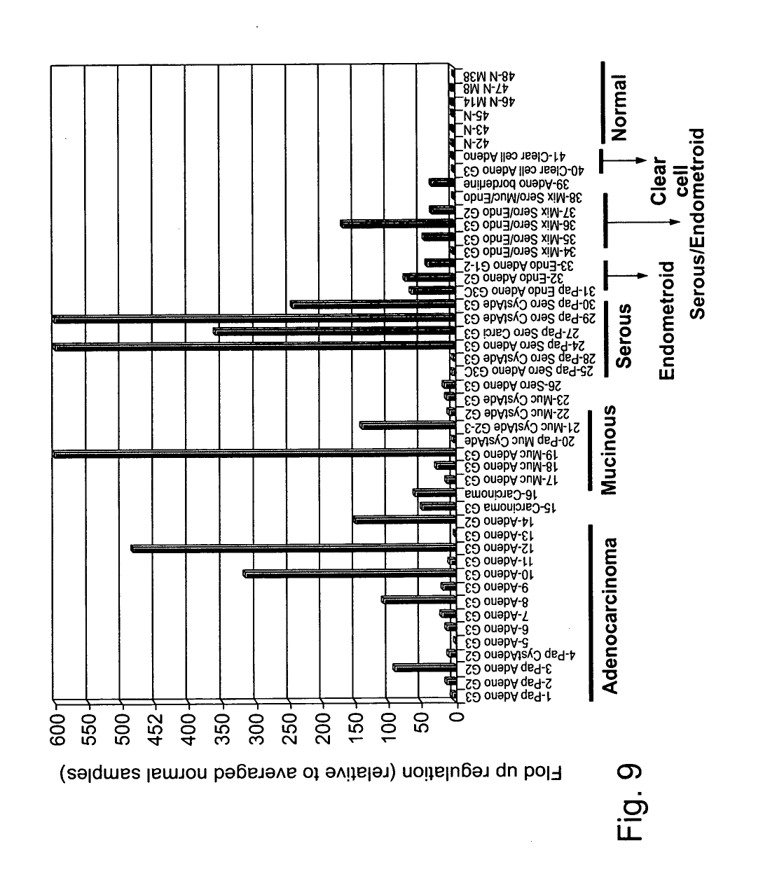

As shown in FIG. 9, SIM2 expression in normal samples (sample Nos. 42-48, Table 2) was significantly lower than in the cancer samples. Notably, the highest expression of SIM2 was found in 4 out of 6 papillary serous samples (carcinoma or adenocarcinoma or cystadenocarcinoma).

### Example 8

### Expression of SIM2Long Variant-Derived Fragment (SEQ ID NO:18) in Normal and Cancerous Lung Tissues

Expression of SIM2 long variant (SEQ ID NO: 7) was measured in normal and cancerous lung tissues (see Table 3, below) by real time PCR using a sequence fragment (SEQ ID NO: 18 corresponding to nucleotide coordinates 1551-1670 of SEQ ID NO: 7 using the primers set forth in SEQ ID NOs: 14-15, designated as primers 12 and 13 in FIG. 1, respectively). In addition the expression of three housekeeping genes—SDHA (GenBank Accession No: NM_{—}004168, SEQ ID NO: 38 was measured using the primers set forth in SEQ ID NOs: 36 and 37), RPS27A (GenBank Accession No: NM_{—}002954, SEQ ID NO: 23 was measured using the primers set forth in SEQ ID NOs: 21, 22), PBGD (GenBank Accession No: HSPBGDR, SEQ ID NO: 32; using the primers set forth in SEQ ID NOs: 30, 31), was measured by real time PCR. In each RT sample, the expression of SIM2 sequences was normalized to the geometric mean of the quantities of the housekeeping genes as detailed in Example 2, hereinabove. The normalized quantity of each RT sample was then divided by the averaged quantity of the normal samples (No. 46-54, Table 3).

TABLE 3

| Serial number | Lot number | Pathology | Source |
|---|---|---|---|
| 1 | A504117 | Adenocarcinoma | Biochain |
| 2 | A504118 | Adenocarcinoma | Biochain |
| 3 | CG-200 | Adenocarcinoma | Ichilov |
| 4 | Com-02-43T-M-2237T | Adenocarcinoma Grade 1 | Clinomics |
| 5 | Com-02-49T-M-2214T | Adenocarcinoma Grade 2 | Clinomics |
| 6 | Com-02-55T-M-2206T | Adenocarcinoma Grade 3 | Clinomics |
| 7 | Com-02-57T-M-2285T | Adenocarcinoma Grade 4 | Clinomics |
| 8 | Com-02-59T-M-2261T | Adenocarcinoma Grade 2 | Clinomics |
| 9 | Com-02-41T-M-2269T | Adenocarcinoma Grade 3 | Clinomics |
| 11 | Com-02-53T-M-2221T | Adenocarcinoma Grade 1 | Clinomics |
| 12 | A504119 | Moderately adenocarcinoma | Biochain |
| 13 | A504116 | moderately to poorly adenocarcinoma | Biochain |
| 14 | CG-111 | Adenocarcinoma | Ichilov |
| 15 | CG-244 | Bronchioloalveolar adenocarcinoma | Ichilov |
| 16 | A409091 | Moderately squamous | Biochain |
| 17 | A503183 | moderately squamous | Biochain |
| 18 | A503387 | moderately to poorly squamous | Biochain |
| 19 | A408175 | Squamous | Biochain |
| 20 | A501121 | Squamous | Biochain |
| 21 | A503187 | Squamous | Biochain |
| 22 | A503386 | Squamous | Biochain |
| 23 | CG-109 (1) | Squamous | Ichilov |
| 24 | CG-123 | Squamous | Ichilov |
| 25 | CG-204 | Squamous | Ichilov |
| 26 | Com-02-47T-M-2208T | Squamous Grade 3 | Clinomics |
| 27 | Com-02-61T-M-2215T | Squamous Grade 2 | Clinomics |
| 28 | Com-02-63T-M-2216T | Squamous Grade 3 | Clinomics |
| 29 | Com-02-65T-M-2239T | Squamous Grade 1 | Clinomics |
| 30 | A501389 | Small cell | Biochain |
| 31 | A501390 | Small cell | Biochain |
| 32 | A501391 | Small cell | Biochain |
| 33 | A504115 | Small cell | Biochain |
| 34 | Com-02-45T-M-2217T | Small cell Grade 2 | Clinomics |
| 35 | Com-02-69T-M-2210T | Small cell Grade 3 | Clinomics |
| 36 | Com-02-71T-M-2218T | Small cell Grade 2 | Clinomics |
| 37 | Com-02-73T-M-2235T | Small cell Grade 1 | Clinomics |
| 38 | A504113 | large cell | Biochain |
| 39 | A504114 | large cell | Biochain |
| 40 | Com-02-75T-M-2212T | Large cell Grade 3 | Clinomics |
| 41 | Com-02-77T-M-2257T | Large cell Grade 4 | Clinomics |
| 42 | Com-02-79T-M-2241T | Large cell Grade 2 | Clinomics |
| 43 | Com-02-163T-M-2290T | Large cell Grade 1 | Clinomics |
| 44 | A501123 | Moderately alveolus carcinoma | Biochain |
| 45 | A501221 | Alveolus carcinoma | Biochain |
| 46 | A501124 | Normal | Biochain |
| 47 | A503205 | Normal | Biochain |
| 48 | A503206 | Normal | Biochain |
| 49 | A503384 | Normal | Biochain |
| 51 | Com-02-44N-M-2237N | normal M4 | Clinomics |
| 53 | Com-02-42N-M-2269N | normal M9 | Clinomics |
| 54 | Com-02-48N-M-2208N | normal M26 | Clinomics |

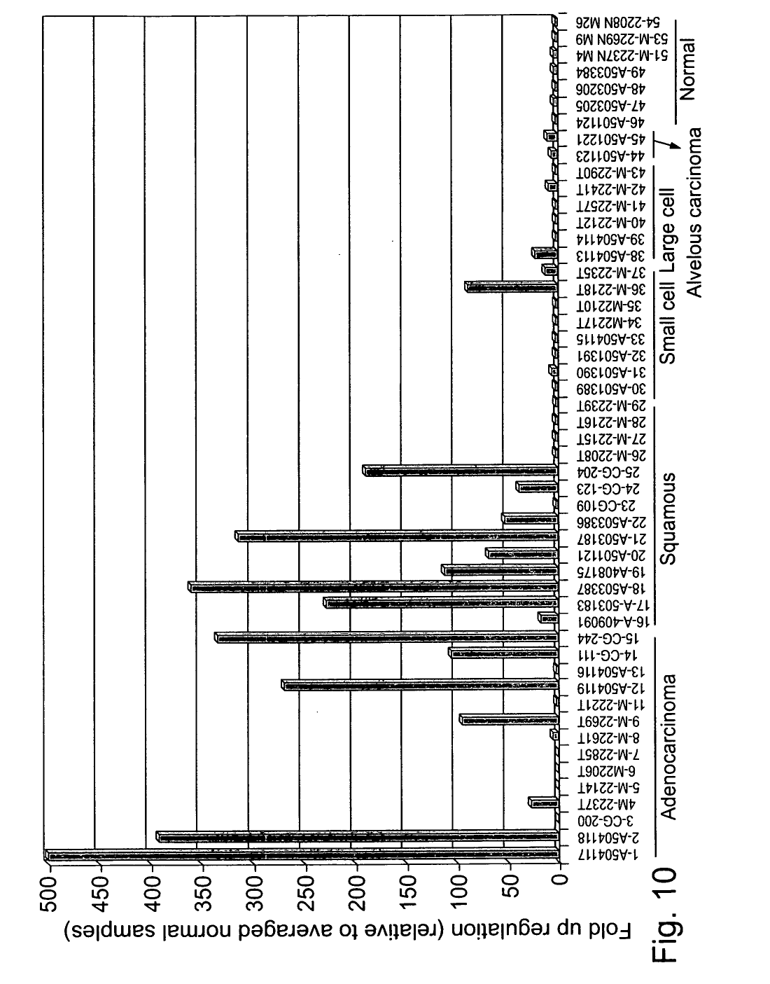

As shown in FIG. 10, SIM2 expression in normal samples (sample Nos. 46-54, Table 3) was significantly lower than in the cancer samples. Interestingly, high expression was found in adenocarcinoma samples (7 out of the 15 samples) and squamous cell carcinoma samples (9 out of the 14 samples).

### Example 9

### Expression of SIM2Short Variant-Derived Fragment (SEQ ID NO:19) in Normal and Cancerous Ovary Tissues

Expression of SIM2 short variant (GenBank Accession No: gi7108361, SEQ ID NO: 8) was measured by real time PCR using the sequence fragment set forth in SEQ ID NO: 19 (nucleotide coordinates 1224-2324 of SEQ ID NO: 7, amplified using the primers set forth in SEQ ID NOs: 16-17 designated as primers 14 and 15 of FIG. 1, respectively). In addition the expression of two housekeeping genes—PBGD was measured by real time PCR as described above. In each RT sample, the expression of SIM2 sequences was normalized to the geometric mean of the quantities of the housekeeping genes as detailed in Example 2, hereinabove. The normalized quantity of each RT sample was then divided by the averaged quantity of the normal samples (No. 42-49, Table 2 above).

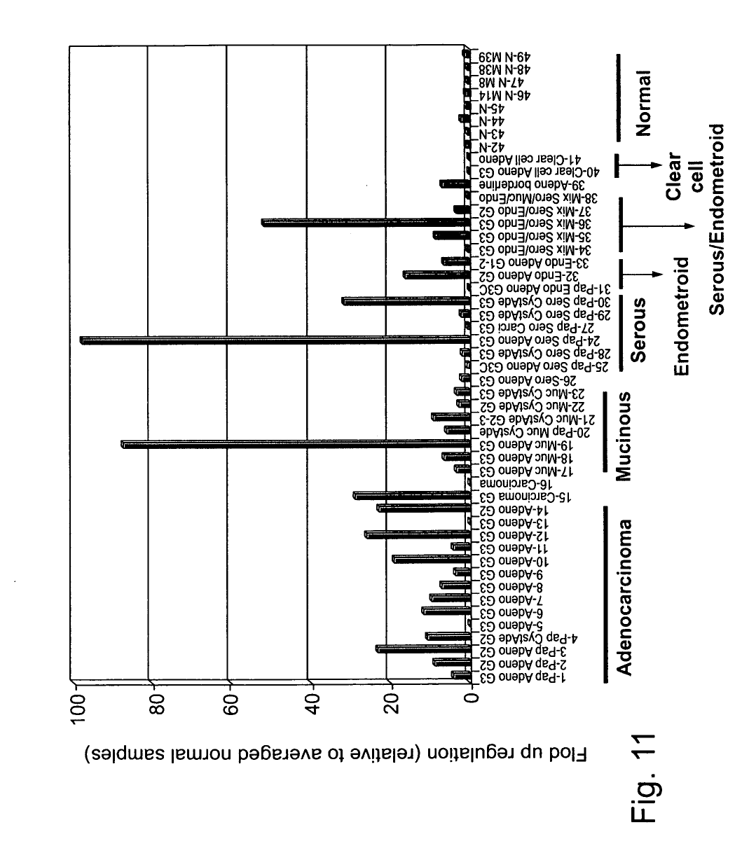

As shown in FIG. 11, SIM2 expression in normal samples (sample Nos. 42-49, Table 2 above) was significantly lower than in the cancer samples.

### Example 10

### Expression of SIM2 Long Variant-Derived Fragment (SEQ ID NO:18) in Normal and Cancerous Breast Tissues

Expression of SIM42 short variant (SEQ ID NO: 7) was evaluated by RT-PCR using a sequence fragment (SEQ ID NO: 18, described above). As shown in FIG. 12, SIM2 expression in most tumor samples was higher than in the normal samples (Table 4, below).

### Example 11

### Expression of SIM2 Short Variant-Derived Fragment (SEQ ID NO:19) in Normal and Cancerous Breast Tissues

Expression of SIM2 short variant (SEQ ID NO: 8) was evaluated by RT-PCR of a sequence fragment (SEQ ID NO: 19, primers 14 and 15, see FIG. 1) normal and cancerous breast samples (see Table 4, below)

TABLE 4

| Serial number | Cat No. | Pathology | Source |
|---|---|---|---|
| 1 | M-0140T | DCIS Grade 1 | clinomics |
| 2 | M-0110T | DCIS Grade 2 | clinomics |
| 3 | M-0150T | IDC Grade 1 | clinomics |
| 4 | M-2159T | IDC Grade 1 | clinomics |
| 5 | M-2168T | IDC Grade 1 | clinomics |
| 6 | 7238T | IDC - G1 | ABS |
| 7 | 7263T | IDC - G2 | ABS |
| 8 | M-0113T | IDC Grade 2 | clinomics |
| 9 | M-0160T | IDC Grade 2 | clinomics |
| 10 | M-2160T | IDC Grade 2 | clinomics |
| 11 | M-2175T | IDC Grade 2 | clinomics |
| 12 | 1432T | IDC - G2 | ABS |
| 13 | A0133T | IDC - G2 | ABS |
| 14 | A0135T | IDC - G2 | ABS |
| 15 | 7259T | IDC - G2 | ABS |
| 16 | 20032T | IDC - G2 | ABS |
| 17 | 20036T | IDC - G2(3) | ABS |
| 18 | M-2169T | IDC Grade 2 | clinomics |
| 19 | M-2162T | IDC Grade 2 | clinomics |
| 20 | M-0111T | IDC Grade 3 | clinomics |
| 21 | M-0112T | IDC Grade 3 | clinomics |
| 22 | M-0114T | IDC Grade 3 | clinomics |
| 23 | M-0115T | IDC Grade 3 | clinomics |
| 24 | M-0180T | IDC Grade 3 | clinomics |
| 25 | M-2176T | IDC Grade 3 | clinomics |
| 26 | 7249T | IDC - G3 | ABS |
| 27 | 20072T | IDC - G3 | ABS |
| 28 | M-2161T | IDC Grade 3 | clinomics |
| 29 | M-2170T | IDC Grade 3 | clinomics |
| 30 | M-2177T | IDC Grade 3 | clinomics |
| 31 | CG-154 | IDC | Ichilov |
| 32 | 7116T | Mucinous carcinoma | ABS |
| 33 | M-0140N | normal matched to 1T | clinomics |
| 34 | M-0110N | normal matched to 2T | clinomics |
| 35 | 7238N | normal matched to 6T | ABS |
| 36 | 7263N | normal matched to 7T | ABS |
| 37 | M-0150N | normal matched to 3T | clinomics |
| 38 | 7116N | normal matched to 32T | ABS |
| 39 | 7259N | normal matched to 15T | ABS |
| 40 | 1432N | normal matched to 12T | ABS |
| 41 | 7249N | normal matched to 26T | ABS |
| 42 | 20031T | IDC Grade 3 | ABS |

IDC = Invasive Ductal Carcinoma
DCIS = Ductal Carcinoma In Situ

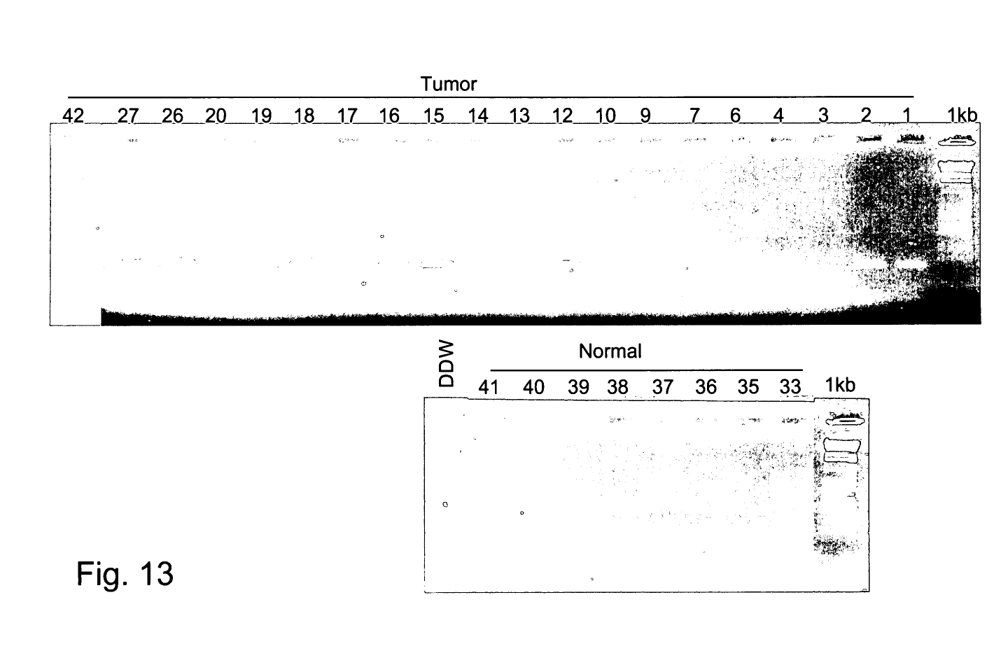

As shown in FIG. 13, SIM2 expression in normal samples (sample Nos. 33-41, Table 4 above) was significantly lower than in the cancer samples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of diagnosing predisposition to, or presence of ovarian cancer, breast cancer and/or lung cancer in a subject, the method comprising determining a level of SIM2 in a biological sample obtained from the subject, said level being correlatable with predisposition to, or presence or absence of the ovarian cancer, breast cancer and/or lung cancer, thereby diagnosing predisposition to, or presence of ovarian cancer, breast cancer and/or lung cancer in the subject, said level of SIM2 being determined according to expression of a polynucleotide sequence according to SEQ ID NO:7 or a fragment thereof.

2. The method of claim 1, wherein said biological sample is a tissue sample and/or a body fluid sample.

3. The method of claim 2, wherein said tissue sample is selected from the group consisting of an ovarian tissue, a lung tissue and a breast tissue.

4. The method of claim 1, wherein said SIM2 fragment comprises SEQ ID NOs: 9.

5. The method of claim 1, wherein said determining level of said SIM2 is effected at an mRNA level.

6. The method of claim 1, wherein said determining level of said SIM2 is effected at a protein level.

7-17. (canceled)

18. Use of a SIM42 detecting agent for detecting ovarian, breast and/or lung cancer.

19. The use of claim 18, wherein said agent for detecting ovarian, breast and/or lung cancer is an oligonucleotide.

20. (canceled)

21. The use of claim 18, wherein said agent for detecting ovarian, breast and/or lung cancer is coupled to a detectable moiety selected from the group consisting of a chromogenic moiety, a fluorogenic moiety, a radioactive moiety and a light-emitting moiety.

22-41. (canceled)

42. The method of claim 5, wherein said determining level of said SIM2 comprises amplification with a primer pair for specifically amplifying a polynucleotide according to SEQ ID NO:7.

43. The method of claim 42, wherein said primer pair is capable of specifically amplifying polynucleotide according to SEQ ID NO:9.

44. The method of claim 43, wherein said primer pair comprises SEQ ID NOs: 10 and 11.

45. The method of claim 1, wherein said lung cancer comprises one of adenocarcinoma or squamous cell carcinoma.

46. The method of claim 1, wherein said ovarian cancer comprises a papillary serous ovarian cancer.

47. The method of claim 46, wherein said papillary serous ovarian cancer comprises one of carcinoma, adenocarcinoma or cystadenocarcinoma.


SEQUENCE LISTING (41 sequences)

SEQ ID NO: 1 - DNA, 1001 bp, Homo sapiens
ggaatattcgaaaccccgagcttttacaacataaagcgcatggtgtggccgcggcgggtaatggcgctctgggagccctgcccaggcggcctctgctcgccctcctccacttccagctccgagctgggtgtgttgcaagtttcatactcctacatattataagtgacactaatatcagggacaactaagtgctggggaacttcaatgaaaacctggctggtaaagtcaacacccccagacttctctgtgctacatttctttaattaattccggagtggtgtgtggacgggcgtctttgcagttattatacacgtaagtgaattaggccatttgaagctacgaagtcatacccaacattttccattaagaatattatttttttagctactgctggcaacttttagaatttaattatgataattttcctcttttcctcattatcccagatatggctggttgtgagatactttttcactaaatgtgtctttttaatgattttggaattaagcaagtatgccaaatgcgccaagacatttataactttagaaattgctgtatagtatatatttttggaacaccacaggtttagttgggaaaatattttgcagctgagttagaaacttgaaagttaggcttataatcaagatgctgattttcaaccttagcatcggggaaggtaatgatagtttagttggcaaagactttttgcagcaaactgtatttgagacagcagaatccaaggatatctttcaagattcacttatactacattctttttagccccctctctaggggtggagggggtggcttagaaaaaccaaaggtaatctggtttcaattacatgctgtaaaaatagaatttgtggccagaaattaatttggaatattttttatgggggcaacattgtgggttgtatgagtctttcaccaactttattgcttttctttggttctggatctaaaatatgaatgagtaaataaaatacagtttcctttttcaa

SEQ ID NO: 2 - DNA, 775 bp, Homo sapiens
tttacaacataaagcgcatggtgtggccgcggcgggtaatggcgctctgggagccctgcccaggcggcctctgctcgccctcctccacttccagctccgagctgggtgtgttgcaagtttcatactcctacatattataagtgacactaatatcagggacaactaagtgctggggaacttcaatgaaaacctggctggtaaagtcaacacccccagacttctctgtgctacatttctttaattaattccggagtggtgtgtggacgggcgtctttgcagttattatacacactttggatggatttgtttttgtggtagcatctgatggcaaaatcatgtatatatccgagaccgcttctgtccatttaggcttatcccaggtggagctcacgggcaacagtatttatgaatacatccatccttctgaccacgatgagatgaccgctgtcctcacggcccaccagccgctgcaccaccacctgctccaagagtatgagatagagaggtcgttctttcttcgaatgaaatgtgtcttggcgaaaaggaacgcgggcctgacctgcagcggatacaaggtcatccactgcagtggctacttgaagatcaggcagtatatgctggacatgtccctgtacgactcctgctaccagattgtggggctggtggccgtgggccagtcgctgccacccagtgccatcaccgagatcaagctgtacagtaacatgttcatgttcagggccagccttgacctgaagctgatattcctggattccag

SEQ ID NO: 3 - DNA, 8296 bp, Homo sapiens
ggggctccgcgggcctggagcacggccgggtctaatatgcccggagccgaggcgcgatgaaggagaagtccaagaatgcggccaagaccaggagggagaaggaaaatggcgagttttacgagcttgccaagctgctcccgctgccgtcggccatcacttcgcagctggacaaagcgtccatcatccgcctcaccacgagctacctgaagatgcgcgccgtcttccccgaaggtgaggcctcaggtgggcggccggggacgctggggagcccggcggccccggcccaggcgggaagcgcaagccagcccgcccagaggggttgccgcggcctggcgtccagagctggggcgtctgagggaggttgcgtgagggtcttcggcttcggcgctggcttggggcgaggggccagggccttggcggcccaggcgaccaaaccctctcctggtccagggctgggtgagggcgaattacgaattgttccaggggcaggcagtcccccagcccgcacggccagcgagcgctgcgagtcagcggggatcacggtgaggcccaagcactgcaggctgaggccacagagcgaacacttgtgctgagccgggccctctcgtgaggctggggtgcgggaagtccgggcaggagagacccgcccccgccgttgctgagctgagacccggctgaaagagaggggtccgattaattcgaaaatggcagacagagctgagcgctgccgttcttttcaggattgaaaatgtgccagtgggccaggggcgctgggacccgcggtgcggaagactcggaacaggaagaaatagtggcgcgctgggtgggctgccccgccgcccacgccggttgccgctggtgacagtggctgcccggccaggcacctccgagcagcaggtctgagcgtttttggcgtcccaagcgttccgggccgcgtcttccagagcctctgctcccagcggggtcgctgcggcctggcccgaaggatttgactctttgctgggaggcgcgctgctcagggttctggtgggtcctctgggcccaggagctgggagggctgcgccggcctctggagccccgggagccagtgccgaggtagggagacaacttccgccgcagggcgccggacggtcggggcagagcaggcgacaggtgtccctaggccgcagggcgcttccatagcgccatccccaccaggcactctactcgaaatcggaaagctcgaccttttgcgttcgcctctgccaagcctgttatttgtgctggccgctgggtctggagctgcgcttctcggcccctccccggtggagcgcagagggctggtctgcaagcgcggcctccagccccgcggctccccggcccaggagccaggcgcgggctgacccgggagcacccggcagcggagggggctggaagcggaccctaggcctctcctgtgccacccggccctaccgcgcggccgcggggcgctctcctctcgggcgcagcggtccttcagcccagggcaggttcctccctttcctactcggaacgtggcaaagataccccagtcccagcccctccagctgagagctgttgcccaaggtcgtcgctacttgtccgctcaatggtgaccccttggcagagaactagggatgattccactccggttgatgttttaggggaaattaaaagaacattcggttttctgagtctccttccggggaggcgtggtggtaactggtttgctgggaagagccgttccttaaccgcatgcaacaaagcagggcgtcagagatgtgaggcaattctctacctccgctggaaaaaatgcagatttattaaaggtcgactgtttagcagaacaacgtagattttttacaacgctttccccgtctctgctttgaagcctgccaggctgcagctggggatccaggagggaaagcccgcaggcgcagaggggacaatccgggaagtggtaaaggggacacccgggcacagggcctgtgctttcgttgcaggcgaggaagtggagcgcgcgctgcagattcagcgcggggctagaggaggggacctggatccctgaaccccggggcggaaagggagcctccgggcggctgtgggtgccgcgctcctcggagccagcagctgctggggcggcgtccgaactccccaggtctgcgcacggcaatgggggcaccgggccttctgtctgtcctcagaatacgtaggatacccgcgggcgacaagccgggccaggctaggagcctccttccctgcccctccccatcggccgcgggaggctttcttggggcgtccccacgaccacccccttctcacccggtccccagtttggaaaaaggcgcaagaagcgggcttttcagggaccccggggagaacacgagggctccgacgcgggagaaggattgaagcgtgcagaggcgccccaaattgcgacaatttactgggatccttttgtggggaaaggaggcttagaggctcaagctataggctgtcctagagcaactaggcgagaacctggccccaaactccctccttacgccctggcacaggttcccggcgactggtgttcccaagggagccccctgagcctaccgcccttgcagggggtcgtgctgcggcttctgggtcataaacgccgaggtcgggggtggcggagctgtagaggctgcccgcgcagaaagctccaggatcccaatatgtgcttgcgtggagcagggagcggaagaggcagccggtcctcaccctcctctcccgccacgcacatatccttcttgacttcgaagtggtttgcaatccgaaagtgagaccttgagtcctcagatggccggcaacgcgccgaggtcacgctccccagaaacacccctctcccctcccctaccccagctccccctggggcgggtggtaattgggggaggagaggccgcaggcagggaaggggtgggaaagccagagagggaggcacaaagtgatggcagcccggcaaacactggggcttcgggctgggccgcgctcgtttaatcccacaaaaatcccattttggaggtgagaaatagaggttagaggtcgggcccttctggagatcagaccgaggagacgggcccagctggcgtcttaaagcaaggagggggagtcgggaggaggaccagcttcggaagaggagggatcgcttggaggccgtgcagtgtgaggaacggcaggcagggtgtgggaccaacatgcacacactcgcaggtgctggggccagggaggaatgaggcgctggctccctttccctccatttctccctgggggtcccagcaacctggccatccctgacttccaacagcacagcgtccccacaggtcctgcagtgctctgcaggggtgcagggagctcccctccccccagccgcaacctcaccttcctcacccccacccctccggcaggaaaccacaggctgggttggggacccctggtgctccaagagagcagtgcagatttctccccaagtcttcccgccatgggctttgcaagaagccagggcccagaggccacgctcaccgttaacactgcacagggcaaaggtggctccaggacaactgcccaaccccaggaacgacccagcagcagagaaaaggacagctgccagggtgcctttgttccccgtccctcgtatcccgcgctgcccgggggctcctgcctttggttcagtgctcgcggcaccaccgcactcaggacggcagtggggggctggggctggggctgggcctggcccagcgtgggttggggcgggggacgcgccagcagcgcccgcagctcgctccgcaggggtcgcagccaggggtcgggagctaggctcgtgggccgggagacgccgggcgcgttgtcctccggggaggttggggtgcaggcggggaagcctggggtctcgcggggcgcagcagtcaggtcgagggtgcagcaggaggggagtcctgacgggcaggtccctctttcccctggtgcgcaacactggttggtagcttttgcggaggtggtgaagaagggcaggaggcctgttgagcggaggagtccggggatccctaattatgtgacaggagaccctttccagttcggcctgtggcccatccctctctcaccgccggcagattggagtctgctctcggggagcccccagccttttctttttaacagagggcaaaggggcgacggcgagagcacagatggcggctgcggagccggggaggcggcggggagacgcgcgggactcgtggggagggctggcagggtgcaggggttccgcgtgacctgcccggctcccaggcatcgggctgggcgctgcagtttaccgatttgctttcgtccctcgtccaggtttaggagacgcgtggggacagccgagccgcgccgggcccctggacggcgtcgccaaggagctgggatcgcacttgctgcaggtagagcggcctcgccgggggaggagcgcagccgccgcaggctcccttcccaccccgccaccccagcctccaggcgtcccttccccaggagcgccaggcagatccagaggctgccgggggctggggatggggtggtccccactgcggagggatggacgcttagcatgtcggatgcggcctgcggccaaccctaccctaaccctacagcccacccggataaccagaacttggtgaggcctccgggctcttgcttggtttggagccaggtgcttagcgccccgagcccggggccattcaccctgcaggagctgcacgcgcccctgacctcggcttttccctggcagcagaggggctttgcgggtcggccgggtagccctgagcacagctcgccacttccaggtgggctgttggcgctggctggggacacatcccgatctttcaaatgccctttacagagcctcatcaacgacccgattcattcccccctcctgtcatttgtctctgccatcgaaaaatgcctaccgagagctgctctgcatttccgccctctattttgtgttttactttaaaataataataaaaaaaatgttggctgcaggacgccatgacttaggtcagcgagtcagccgctagctctgcatttccaaaaagcagatcttttcacaactctcttgccccaagtgccctggtgtggtttattttttaaaatgcatgcctgcggaagagaagacccggggaatattcgaaaccccgagcttttacaacataaagcgcatggtgtggccgcggcgagtaatggcgctctgggagccctgcccaggcggcctctgctcgccctcctccacttccagctccgagctgggtgtgttgcaagtttcatactcctacatattataagtgacactaatatcagggacaactaagtgctggggaacttcaatgaaaacctggctggtaaagtcaacacccccagacttctctgtgctacatttctttaattaattccggagtggtgtgtggacgggcgtctttgcagttattatacacgtaagtgaattaggccatttgaagctacgaagtcatacccaacattttccattaagaatattatttttttagctactgctggcaacttttagaatttaattatgataattttcctcttttcctcattatcccagatatggctggttgtgagatactttttcactaaatgtgtctctttaatgattttggaattaagcaagcatgccaaatgcgccaagacatttataactttagaaattgctgtatagtatatatttttggaacaccacaggtttagttgggaaaatattttgcagctgagttagaaacttgaaagttaggcttataatcaagatgctgattttcaaccttagcatcggggaaggtaatgatagtttagttggcaaagactttttgcagcaaactgtatttgagacagcagaatccaaggatatctttcaagattcacttatactacattctttttagccccctctctaggggtggagggggtggcttagaaaaaccaaaggtaatctggtttcaattacatgctgtaaaaatagaatttgtggccagaaattaatttggaatattttttatgggggcaacattgtgggttgtatgagtctttcaccaactttattgcttttctttggttctggatctaaaatatgaatgagtaaataaaatacagtttcctttttcaaaaataatttagtctttttatccttgacacataaatataatcattacctttttatttgtcttaattacctcttatagttttactttatttttgagggttagccaacttaggaaaaatcagaatttcatttgggtagttttcatttttcagaagttgatacttttcctccttgaattttgcaaaggttatttgatctcttgcaaattctaaataattaattctgtagacatagtctggtaaaataaccaggtattttgttttattttctgggcagtggtagtgttcggaatttattattccaacggtacttagaggtaactagaaaattgtggcaaataaagcatatgtcctagtggtataaatgactgccaattatgacataaaatggtctatatagtggtagattttcccctaagtaccatagaactccggatttgtactaagtgctaaaataaccccaaacaataaagctactacgtttaacctgagcttgataggaattctcaaatgttttgcatgatttttgtgatgtggatgtatgattgtttctaaagtgctttaaaagcatttcagagcctggcctgggaactaaaccctgggtgctgtttcctacagtcctttttaagaccctgggcttcatttttgcatttagcaagtatgtgttgatggtcatatttgatgcatccctccatcccagtgactgtaccattgttagatttaactctaatgtagaatgatcatctcttattctgacactttatcttttacagactttggatggatttgtttttgtggtagcatctgatggcaaaatcatgtatatatccgagaccgcttctgtccatttaggcttatcccaggtgggtattgcctaattttatgtgcaaccaaaatattaaacgaagtgacagcagattttgacagcctcacccaacttgaaaatgagtctgtttaagtgtttgcttttgtgtaagaacataaccctaaattgcaaaatcctcttctcaggacatcctgcaggggttttcttctccatattacgttttacttttttactttctttttcccttggggtttcatgaccagtgtgtcttcaccccgtcagccttgttccgagattctgccaaaacccggactatctgcttatgtagtaatgctagatgccctgaaaagagctccacaaagggacatgtttcagtccacacttgctggtctgcacgttacggtattatgagttgctataaagcaagaggtaagctgatgtggaacatggattttatgcagaatgttctttggggagagagaatggggaggggtagatttatgagtgttcgcatggaattcggggtcatttttcctgactttttccagttcagtggtcgtttcagcaaagcattttatatgcttgtgatagaaactcataaaactcagtttactactgttttgaacagcaaggtttctttagaacatctgaatattaccaatttttctggaaagatggaatcatcttcttaaaaaagtccaccgtttccttagaggaaagagggggactcagctttgtctctccctggcaagccaagggtttgatgggggatttgggggctccctgggcccattgaatgtctgaaaggggatagtgtaactgatcattctcattcacatgcctgacttaacaaatgtggatcatgattttaaaagtcacattttaaactacatatagggactaaatatttcttcacattgtgtactatcagagtgatttttcaccgtgactgaagacacactagaattaatgatagatacagaggatgggttccagagaggcccatattgaatttgaattacacctgcctcctaatgtagttaagccaaaataaaacatgccgattaatgtggcttcagtttgctcattgaaaaacaaaatgaccacatttggcaaactgcatctggtgtattccttttatttttctgccaatacttttttttataagcttgctatttatttacatccttctcttctgatgtatggatatccttatccagatccacatgggtttatgtaactctgtgtttctcaggcaaaaccctttaaaatgaattacatattaaaatgatatggaag

SEQ ID NO: 4 - DNA, 23 bp, Artificial sequence
  Single strand DNA oligonucleotide
tgggtgtgttgcaagtttcatac

SEQ ID NO: 5 - DNA, 23 bp, Artificial sequence
  Single strand DNA oligonucleotide
ctttaccagccaggttttcattg

SEQ ID NO: 6 - DNA, 22 bp, Artificial sequence
  Single strand DNA oligonucleotide
ccttggattctgctgtctcaaa

SEQ ID NO: 7 - DNA, 3885 bp, Homo sapiens
ggggctccgcgggcctggagcacggccgggtctaatatgcccggagccgaggcgcgatgaaggagaagtccaagaatgcggccaagaccaggagggagaaggaaaatggcgagttttacgagcttgccaagctgctcccgctgccgtcggccatcacttcgcagctggacaaagcgtccatcatccgcctcaccacgagctacctgaagatgcgcgccgtcttccccgaaggtttaggagacgcgtggggacagccgagccgcgccgggcccctggacggcgtcgccaaggagctgggatcgcacttgctgcagactttggatggatttgtttttgtggtagcatctgatggcaaaatcatgtatatatccgagaccgcttctgtccatttaggcttatcccaggtggagctcacgggcaacagtatttatgaatacatccatccttctgaccacgatgagatgaccgctgtcctcacggcccaccagccgctgcaccaccacctgctccaagagtatgagatagagaggtcgttctttcttcgaatgaaatgtgtcttggcgaaaaggaacgcgggcctgacctgcagcggatacaaggtcatccactgcagtggctacttgaagatcaggcagtatatgctggacatgtccctgtacgactcctgctaccagattgtggggctggtggccgtgggccagtcgctgccacccagtgccatcaccgagatcaagctgtacagtaacatgttcatgttcagggccagccttgacctgaagctgatattcctggattccagggtgaccgaggtgacgggttacgagccgcaggacctgatcgagaagaccctataccatcacgtgcacggctgcgacgtgttccacctccgctacgcacaccacctcctgttggtgaagggccaggtcaccaccaagtactaccggctgctgtccaagcggggcggctgggtgtgggtgcagagctacgccaccgtggtgcacaacagccgctcgtcccggccccactgcatcgtgagtgtcaattatgtactcacggagattgaatacaaggaacttcagctgtccctggagcaggtgtccactgccaagtcccaggactcctggaggaccgccttgtctacctcacaagaaactaggaaattagtgaaacccaaaaataccaagatgaagacaaagctgagaacaaacccttaccccccacagcaatacagctcgttccaaatggacaaactggaatgcggccagctcggaaactggagagccagtccccctgcaagcgctgctgctcctccagaactgcagccccactcagaaagcagtgaccttctgtacacgccatcctacagcctgcccttctcctaccattacggacacttccctctggactctcacgtcttcagcagcaaaaagccaatgttgccggccaagttcgggcagccccaaggatccccttgtgaggtggcacgctttttcctgagcacactgccagccagcggtgaatgccagtggcattatgccaaccccctagtgcctagcagctcgtctccagctaaaaatcctccagagccaccggcgaacactgctaggcacagcctggtgccaagctacgaagcgcccgccgccgccgtgcgcaggttcggcgaggacaccgcgcccccgagcttcccgagctgcggccactaccgcgaggagcccgcgctgggcccggccaaagccgcccgccaggccgcccgggacggggcgcggctggcgctggcccgcgcggcacccgagtgctgcgcgcccccgacccccgaggccccgggcgcgccggcgcagctgcccttcgtgctgctcaactaccaccgcgtgctggcccggcgcggaccgctggggggcgccgcacccgccgcctccggcctggcctgcgctcccggcggccccgaggcggcgaccggcgcgctgcggctccggcacccgagccccgccgccacctccccgcccggcgcgcccctgccgcactacctgggcgcctcggtcatcatcaccaacgggaggtgacccgctggccgcccgcgccaggagcctggacccggcctcccggggctgcggcgccaccgagcccggcaaatgcgcacgacctacattaatttatgcagagacagctgtttgaattggaccccgccgccgacttgcggatttccaccgcggaggccccgcgcgccggtgccgagggccgaggagcgcccgggtccgggcaggtgaccgcccgcctctgtcctgcgagggccggtgcgacccagttgctgggggcttggtttcctcaccttgaaatcgggcttcacgcgtcttgccttgtccccaacgttccacaacagtcccgctgggggattgaagcggtttcactccgcaaatatcctccactttcaggagggaaaacccaccctaccacagtccgctcttccaagtggacggcagacctgggaggggacgcctgtgtcacgagcccttttagatgcttaggtgaaggcagaagtgatgattgtaagtcccatgaatacacaactccactgtctttaaaagtcattcaagagtctcattatttttgtttttatttaaccctttcttcaatacaaaaagccaacaaaccaagactaagggggtgaccatgcaattccattttgtgtctgtgaacataggtgtgcttcccaaatacattaacaagctcttacttccccctaacccctatgaactcttgataacaccaagagtagcaccttcagaatatattgaataggcattaaatgcaaaaatatatatgtagccagacagtttatgagaatgaccctgtcaagcttcattattacgtggcaaaatccctctggcccacacagatctgtaattcactaggctcgtgtttgctacaaatagtgctaataaagttaaattgcacgtgcaatacggaacactgtcaatggactgcaccttgtgaaggaaaaacatgcttaagggggtgtaatgaaaatgatgtagacattttaagcattttctacacagcgagaaaacttcgtaagaacatgttacgtgtgcaacaggtaaacagaaatcctttcataaagcaccagcagtgtttaaaaaatgagcttccattaatttttactttttatgggttttgcttaaagatctcaacatggaaaaatcctgtcatggctctgaactgcacaatgcattgaaccgccgtccttcaattttcttcacactatcaacactgcagcattttgctgctttatcaaaatggtttattttaggaaactttttccacctttctgaatggaaagaggttttcacaaatgttttaaactcatcgttctaaaatcaagtgcacctacaccaactgctctcaaaatgtgaactgacttttttttttttttttttgccaaccctgtgtcacttagtgaggacctgacacaatccctacagggtgtctgtcagtgggcctcatggtaagagtcacaatttgcaaatttaggaccgtgggtcatgcagcgaaggggctggatggtaggaagggatgtgcccgcctctccacgcactcagctatacctcattcacagctccttgtgagtgtgtgcacaggaaataagccgagggtattatttttttatgttcatgagtcttgtaattaaaccgtgattcttgaaaggtgtaggtttgattactaggagataccaccgacatttttcaataaagtactgcaaaatgcttttgtgtctaccttgttattaacttttggggctgtatttagtaaaaataaatcaaggctatcggagcagttcaataacaaaggttactgttgagaaaaaagaccctatcatagatttacaag

SEQ ID NO: 8 - DNA, 2823 bp, Homo sapiens
ggggctccgcgggcctggagcacggccgggtctaatatgcccggagccgaggcgcgatgaaggagaagtccaagaatgcggccaagaccaggagggagaaggaaaatggcgagttttacgagcttgccaagctgctcccgctgccgtcggccatcacttcgcagctggacaaagcgtccatcatccgcctcaccacgagctacctgaagatgcgcgccgtcttccccgaaggtttaggagacgcgtggggacagccgagccgcgccgggcccctggacggcgtcgccaaggagctgggatcgcacttgctgcagactttggatggatttgtttttgtggtagcatctgatggcaaaatcatgtatatatccgagaccgcttctgtccatttaggcttatcccaggtggagctcacgggcaacagtatttatgaatacatccatccttctgaccacgatgagatgaccgctgtcctcacggcccaccagccgctgcaccaccacctgctccaagagtatgagatagagaggtcgttctttcttcgaatgaaatgtgtcttggcgaaaaggaacgcgggcctgacctgcagcggatacaaggtcatccactgcagtggctacttgaagatcaggcagtatatgctggacatgtccctgtacgactcctgctaccagattgtggggctggtggccgtgggccagtcgctgccacccagtgccatcaccgagatcaagctgtacagtaacatgttcatgttcagggccagccttgacctgaagctgatattcctggattccagggtgaccgaggtgacgggttacgagccgcaggacctgatcgagaagaccctataccatcacgtgcacggctgcgacgtgttccacctccgctacgcacaccacctcctgttggtgaagggccaggtcaccaccaagtactaccggctgctgtccaagcggggcggctgggtgtgggtgcagagctacgccaccgtggtgcacaacagccgctcgtcccggccccactgcatcgtgagtgtcaattatgtactcacggagattgaatacaaggaacttcagctgtccctggagcaggtgtccactgccaagtcccaggactcctggaggaccgccttgtctacctcacaagaaactaggaaattagtgaaacccaaaaataccaagatgaagacaaagctgagaacaaacccttaccccccacagcaatacagctcattccaaatggacaaactggaatgcggccagctcggaaactggagagccagtccccctgcaagcgctgctgctcctccagaactgcagccccactcagaaagcagtgaccttctgtacacgccatcctacagcctgcccttctcctaccattatggacacttccctctggactctcacttcttcagcagcaaaaagccaatgttgccggccaagttcgggcagccccaaggatccccttgtgaggtggcacgctttttcctgagcacaatgccagccagcggtgaatgccagtggcattatgccaaccccctagtgcctagcagctcgtctccagctaaaaatcctccagagccaccggcgaacactgctaggcacagcctggtgccaagctacgaaggtgggtcaggtctgctcgtggggaaggtgggaggactgcgcacggccgggagccgaagcagccatggcggtgggtggcagatggagacagaaccctcacgctttgggcaaacttgccctctttctgcttctaagtagggcttgctgtgctttcttgctctcaatgcaggtgctcctcgagagtgagaaatggcagtctgcctgcctcggggacactagtgacagtataaagggcaaaggaaaaccgagtatctggccttcacgtaaatcctggccacattcaccaaccaaagggggacagtgattttcaaaaccagctcccatgtgctgagaacaccccagctgcatttcttttgcaagattcctttccactccaaccagaagtgaatatttgagacaaacggcctattggctattttcccatgccagttttggaagtggggaaaactatggtggaaatttgtgggcttggggacagaaatgccactcaccaacccagggcaaagaacacaaaccctccaggcctcagtttcttcacctgtaaaatggggtgaagctgtgatgtgcctactcccaaggacacgacacacagtagggacctgccctgtacatgctagttcaacagaaaggaatggcctttcaccttctcctggtggcaggcaagcagatgtcctctgcggagataccgccagctccccaggacgcagactgactcctgtttgctcgctggaccaaccccaggcagaaggtggaaggtgggaacagaggtttagctgcaggacatgtattcccattgcaccgagacctaactgccgctcagagtgtagaccgagatggtgcagatgcctgcagtgccattaaaatgtgggtgaaggtgacatcaggattatgtgccccaggccgggctcagtggctcacacctgtaatcccagcactttgggaggccaaggtgggcggatcacctgaggtcaggagtttgcgacaagcctgccaacaagctgaaaccccatctccactaaaaatacaaaaattagttgggcatggtggtgagcacctgtaatcccagctactctggaggctgagataggaggatcacttgaacccgggaggtggaggttgcagtgagctaagatcacatcactgcactccagcctgggtaacagagtgagactgtctcaaaaaaaaaaaaaaa

SEQ ID NO: 9 - DNA, 99 bp, Artificial sequence
  Real time PCR amplicon
aggagctgggatcgcacttgctgcagactttggatggatttgtttttgtggtagcatctgatggcaaaatcatgtatatatccgagaccgcttctgtcc

SEQ ID NO: 10 - DNA, 19 bp, Artificial sequence
  Single strand DNA oligonucleotide
ggagctgggatcgcacttg

SEQ ID NO: 11 - DNA, 21 bp, Artificial sequence
  Single strand DNA oligonucleotide
ggacagaagcggtctcggata

SEQ ID NO: 12 - DNA, 23 bp, Artificial sequence
  Single strand DNA oligonucleotide
tttacaacataaagcgcatggtg

SEQ ID NO: 13 - DNA, 22 bp, Artificial sequence
  Single strand DNA oligonucleotide
gtggctacttgaagatcaggca

SEQ ID NO: 14 - DNA, 20 bp, Artificial sequence
  Single strand DNA oligonucleotide
cctagcagctcgtctccagc

SEQ ID NO: 15 - DNA, 18 bp, Artificial sequence
  Single strand DNA oligonucleotide
ggtgtcctcgccgaacct

SEQ ID NO: 16 - DNA, 21 bp, Artificial sequence
  Single strand DNA oligonucleotide
tagggacctgccctgtacatg

SEQ ID NO: 17 - DNA, 17 bp, Artificial sequence
  Single strand DNA oligonucleotide
gctggcggtatctccgc

SEQ ID NO: 18 - DNA, 120 bp, Artificial sequence
  Real time PCR amplicon
cctagcagctcgtctccagctaaaaatcctccagagccaccggcgaacactgctaggcacagcctggtgccaagctacgaagcgcccgccgccgccgtgcgcaggttcggcgaggacacc

SEQ ID NO: 19 - DNA, 101 bp, Artificial sequence
  Real time PCR amplicon
tagggacctgccctgtacatgctagttcaacagaaaggaatggcctttcaccttctcctggtggcaggcaagcagatgtcctctgcggagataccgccagc

SEQ ID NO: 20 - DNA, 101 bp, Artificial sequence
  Real time PCR amplicon
tgggtgtgttgcaagtttcatactcctacatattataagtgacactaatatcagggacaactaagtgctggggaacttcaatgaaaacctggctggtaaag

SEQ ID NO: 21 - DNA, 20 bp, Artificial sequence
  Single strand DNA oligonucleotide
ctggcaagcagctggaagat

SEQ ID NO: 22 - DNA, 23 bp, Artificial sequence
  Single strand DNA oligonucleotide
tttcttagcaccaccacgaagtc

SEQ ID NO: 23 - DNA, 101 bp, Artificial sequence
  Real time PCR amplicon
ctggcaagcagctggaagatggacgtactttgtctgactacaatattcaaaaggagtctactcttcatcttgtgttgagacttcgtggtggtgctaagaaa

SEQ ID NO: 24 - DNA, 25 bp, Artificial sequence
  Single strand DNA oligonucleotide
cagtgattataggctttcgctctaa

SEQ ID NO: 25 - DNA, 23 bp, Artificial sequence
  Single strand DNA oligonucleotide
cagggctattggttgaatgagta

SEQ ID NO: 26 - DNA, 134 bp, Artificial sequence
  Real time PCR amplicon
cagtgattataggctttcgctctaagattaaaaatgccctagcccacttcttaccacaaggcacacctacaccccttatccccatactagttattatcgaaaccatcagcctactcattcaaccaatagccctg

SEQ ID NO: 27 - DNA, 20 bp, Artificial sequence
  Single strand DNA oligonucleotide
gtaacccgttgaaccccatt

SEQ ID NO: 28 - DNA, 20 bp, Artificial sequence
  Single strand DNA oligonucleotide
ccatccaatcggtagtagcg

SEQ ID NO: 29 - DNA, 151 bp, Artificial sequence
  Real time PCR amplicon
gtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatgg

SEQ ID NO: 30 - DNA, 19 bp, Artificial sequence
  Single strand DNA oligonucleotide
tgagagtgattcgcgtggg

SEQ ID NO: 31 - DNA, 21 bp, Artificial sequence
  Single strand DNA oligonucleotide
ccagggtacgaggctttcaat

SEQ ID NO: 32 - DNA, 91 bp, Artificial sequence
  Real time PCR amplicon
tgagagtgattcgcgtgggtacccgcaagagccagcttgctcgcatacagacggacagtgtggtggcaacattgaaagcctcgtaccctgg

SEQ ID NO: 33 - DNA, 21 bp, Artificial sequence
  Single strand DNA oligonucleotide
tgacactggcaaaacaatgca

SEQ ID NO: 34 - DNA, 21 bp, Artificial sequence
  Single strand DNA oligonucleotide
ggtccttttcaccagcaagct

SEQ ID NO: 35 - DNA, 94 bp, Artificial sequence
  Real time PCR amplicon
tgacactggcaaaacaatgcagactttgctttccttggtcaggcagtataatccaaagatggtcaaggtcgcaagcttgctggtgaaaaggacc

SEQ ID NO: 36 - DNA, 20 bp, Artificial sequence
  Single strand DNA oligonucleotide
tgggaacaagagggcatctg

SEQ ID NO: 37 - DNA, 22 bp, Artificial sequence
  Single strand DNA oligonucleotide
ccaccactgcatcaaattcatg

SEQ ID NO: 38 - DNA, 86 bp, Artificial sequence
  Real time PCR amplicon
tgggaacaagagggcatctgctaaagtttcagattccatttctgctcagtatccagtagtggatcatgaatttgatgcagtggtgg

SEQ ID NO: 39 - protein, 160 aa, Homo sapiens
LDGFVFVVASDGKIMYISETASVHLGLSQVELTGNSIYEYIHPSDHDEMTAVLTAHQPLHHHLLQEYEIERSFFLRMKCVLAKRNAGLTCSGYKVIHCSGYLKIRQYMLDMSLYDSCYQIVGLVAVGQSLPPSAITEIKLYSNMFMFRASLDLKLIFLDS

SEQ ID NO: 40 - protein, 178 aa, Homo sapiens
MKEKSKNAAKTRREKENGEFYELAKLLPLPSAITSQLDKASIIRLTTSYLKMRAVFPEGEASGGRPGTLGSPAAPAQAGSASQPAQRGCRGLASRAGASEGGCVRVFGFGAGLGRGARALAAQATKPSPGPGLGEGELRIVPGAGSPPARTASERCESAGITVRPKHCRLRPQSEHLC

SEQ ID NO: 41 - protein, 584 aa, Homo sapiens
MRRWLPFPPFLPGGPSNLAIPDFQQHSVPTGPAVLCRGAGSSPPPSRNLTFLTPTPPAGNHRLGWGPLVLQESSADFSPSLPAMGFARSQGPEATLTVNTAQGKGGSRTTAQPQERPSSREKDSCQGAFVPRPSYPALPGGSCLWFSARGTTALRTAVGGWGWGWAWPSVGWGGGRASSARSSLRRGRSQGSGARLVGRETPGALSSGEVGVQAGKPGVSRGAAVRSRVQQEGSPDGQVPLSPGAQHWLVAFAEVVKKGRRPVERRSPGIPNYVTGDPFQFGLWPIPLSPPADWSLLSGSPQPFLFNRGQRGDGESTDGGCGAGEAAGRRAGLVGRAGRVQGFRVTCPAPRHRAGRCSLPICFRPSSRFRRRVGTAEPRRAPGRRRQGAGIALAAGRAASPGEERSRRRLPSHPATPASRRPFPRSARQIQRLPGAGDGVVPTAEGWTLSMSDAACGQPYPNPTAHPDNQNLVRPPGSCLVWSQVLSAPSPGPFTLQELHAPLTSAFPWQQRGFAGRPGSPEHSSPLPGGLLALAGDTSRSFKCPLQSLINDPIHSPLLSFVSAIEKCLPRAALHFRPLFCVLL